(19) United States
(12) Patent Application Publication
(10) Pub. No.: US 2006/0128610 A1
(43) Pub. Date: Jun. 15, 2006

(54) Methods of treatment

(75) Inventor: Garth James Smith Cooper, Auckland (NZ)

Correspondence Address: DUANE MORRIS LLP, 101 WEST BROADWAY, SUITE 900, SAN DIEGO, CA, 92101-8285, US

(73) Assignee: Protemix Corporation Limited

(21) Appl. No.: 11/297,169

(22) Filed: Dec. 8, 2005

Related U.S. Application Data
(60) Provisional application No. 60/635,044, filed on Dec. 10, 2004.

(51) Int. Cl. A61K 38/16

(52) U.S. Cl. 514/8

(57) ABSTRACT

The inventions relate generally to methods for the prevention and treatment of conditions, diseases and disorders that would be improved, eased, or lessened by the administration of, for example, a composition comprising a therapeutically effective amount a glyponectin polypeptide, agonist, and/or nucleic acid constructs of the invention.

### CROSS REFERENCE TO RELATED APPLICATIONS

The instant application contains a Sequence Listing which has been submitted via CD-R in lieu of a printed paper copy, and is hereby incorporated by reference in its entirety. Said CD-R, recorded on Dec. 6, 2005, are labeled CRF, “Copy 1.” and “Copy 2“, respectively, and each contains only one identical 21.9 Kb file (36910198.APP).

This application claims the benefit of priority of U.S. Provisional Application No. 60/635,044, filed on Dec. 10, 2004, which is incorporated herein by reference in its entirety.

### FIELD

The inventions relate generally to methods for the prevention and treatment of conditions, diseases and disorders that would be improved, eased, or lessened by the administration of, for example, a composition comprising polypeptides and/or nucleic acid and polynucleotide constructs or other compounds of the invention. Methods of treatment include, for example, methods of treating various alcohol-related diseases, disorders, and conditions.

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art.

Use of adiponectin and glycosylated adiponectin has been discussed in the context of the treatment of mammals for liver disease and TNF-α related diseases. See, e.g., U.S. Pat. App. No. 20040023854. It was reported that circulating concentrations of glyponectin decreased following chronic consumption of high fat ethanol-containing food. Xu, A. et al., Journal of Clinical Investigation 112: 91-100 (2003).

Acute and chronic alcohol intake can have devastating effects on the human body. Chronic alcohol intake causes damage in a number of tissues and organs. Although treatment is available for some alcohol-induced conditions, all of the current treatments have shortcomings, and individuals suffering from the many ramifications of alcohol intake are in need of effective treatment.

Acute intoxication is caused by consumption of large amounts of alcohol over a short period of time. Oscar-Berman, M., and Marinkovic, K., Alcohol Research & Health 27(2): 125-133 (2003). This is also commonly referred to as binge drinking. Acute intoxication is a prevailing epidemic in the United States. In 2001, there were nearly 1.5 billion episodes of binge drinking, of which many reported driving impaired. Centers for Disease Control and Prevention Alcohol Factsheet, General Alcohol Information (2004).

Chronic alcohol consumption involves uncontrolled alcohol intake. Chronic alcohol consumption causes damage to nearly every organ in the body.

Acute and chronic alcohol consumption can lead to chronic alcohol intoxication, alcohol abuse, alcoholism, and alcohol dependence. All conditions have serious detrimental effects and are difficult to treat.

Alcohol-related problems are widespread and affect millions of Americans. Nearly 14 million Americans abuse alcohol or suffer from alcoholism. National Institute on Alcohol Abuse and Alcoholism, Alcoholism-Getting the Facts, Booklet Publication No. 96-4153 (2001). Every year alcohol is responsible for thousands of highway deaths and injuries, suicide, loss of work hours, and many other life threatening problems, and the economic costs of alcohol abuse and alcoholism are astronomical.

A recent estimate of the overall economic cost of alcohol abuse for 1998 was $185 billion. National Institute on Alcohol Abuse and Alcoholism, Alcohol Alert, No. 51 (2001). The overall cost projected for 1998 can be divided into the following categories: $134.2 billion due to lost productivity, which includes $87.6 billion due to losses from alcohol-related illness; $36.5 billion for premature death; crime at a cost of $10.1 billion; $26.3 billion for health care expenditures, which includes $7.5 billion for the treatment of alcohol abuse and dependence and $18.9 billion for the medical consequences of alcohol intake; property and administrative costs of alcohol-related auto-accidents cost $15.7 billion; and $6.3 billion was the cost of service of the criminal justice system. Id.

Evidence indicates that the mind and brain are susceptible to damage from alcohol intake. Alcohol affects the brain's neurons in several ways. It alters neuron membranes, ion channels, enzymes, and receptors. Alcohol also affects several neurotransmitter systems in the brain, including opiates GABA, glutamate, serotonin, and dopamine.

Alcohol affects neurotransmitter systems by decreasing the excitatory actions of the neurotransmitter glutamate and simultaneously increasing the inhibitory actions of the neurotransmitter gamma aminobutyric acid (GABA). Alcohol also helps to increase the release of dopamine, by a process that is still poorly understood but that appears to involve curtailing the activity of the enzyme that breaks down dopamine.

GABA's effect is to reduce neural activity by allowing chloride ions to enter the post-synaptic neuron. These ions have a negative electrical charge, which helps to make the neuron less excitable. This physiological effect is amplified when alcohol binds to the GABA receptor, because it is believed to cause the ion channel to stay open longer and thus let more Cl- ions into the cell.

Alcohol also inhibits excitatory actions of the neurotransmitter glutamate at the NMDA subtype of glutamate receptor. Chronic consumption of alcohol results in the synthesis of more glutamate receptors to compensate for this inhibition. When alcohol is withdrawn, the central nervous system experiences increased excitability to glutamate. This increase in glutamate excitability, plus the desensitized GABAergic receptors, is believed to cause the symptoms of alcohol withdrawal.

Alcohol withdrawal occurs when one regularly consumes alcohol and either dramatically reduces intake or abstains from alcohol consumption altogether. Alcohol withdrawal symptoms can be mild to severe and may even cause death. Kelly, A., RN 67(2): 27-31 (2004). Initial, short-term symptoms of alcohol withdrawal include headache, agitation, anxiety, nausea, vomiting and disorientation. Id. More serious symptoms include hallucinations and delirium tremens. Delirium tremens causes severe agitation, disorientation, tremors, hallucinations, increased heart rate and blood pressure. Id.

Medication for treatment of alcohol withdrawal syndrome includes anticonvulsants such as carbamazepine and valproate. Anton, R., Swift, R., The American Journal on Addictions 12(1): S53-S68 (2003). Side effects include chest pain; high blood pressure; liver damage; numbness in hands, feet, legs or arms; confusion; severe vomiting or nausea; and decreased urination. Valproate can be efficacious; however, a black box warning for life threatening pancreatitis has been added to its side effect profile.

Benzodiazepines are also used to reduce the severity of alcohol withdrawal, including seizures. Kelly, A., RN 67(2): 27-31 (2004). Benzodiazepines are usually safe, but they should not be administered for a long period of time because they can become addictive. Certain types of benzodiazepines have a slower onset and have a lower tendency for abuse than those with a rapid onset of action. Id.

Other medications such as beta-blockers and alpha-adrenergic agonists are used to help alleviate the symptoms of alcohol withdrawal. However, these agents are generally recommended for use in combination with a benzodiazepine. Id. Shortcomings of beta-blockers include delirium as a possible side effect, and they are not known for reducing seizures. Although alpha-adrenergic agonists reduce symptoms of alcohol withdrawal, their efficiency on seizures and delirium is unknown. Id.

Neuroleptic agents, including, for example, the phenothiazines and the butryphenone haloperidol, are another choice for treating alcohol withdrawal symptoms, but are not as effective as benzodiazepines and have been shown to increase the occurrence of seizures.

Alcohol's addictive nature and its rewarding effects are due mostly in part to neurotransmitters such as dopamine, serotonin and opioid peptides. Id. Alcohol increases the release of dopamine and causes serotonin receptor subtypes to stimulate dopaminergic activity. Id. Opioid peptide activity results in a feeling of euphoria and contributes to the rewarding effect of alcohol. Id.

There are no known medicinal treatments to cure alcoholism or alcohol dependency. Rather, medications focus on reducing alcohol consumption and preventing relapse. Their efficacy is said to be modest at best. Disulfiram and naltrexone reduce the pleasurable effects of alcohol and increase the unpleasant effects, such as nausea and vomiting in that upon consumption one becomes ill. Anton, R., Swift, R., The American Journal on Addictions 12(1): S53-S68 (2003). Disulfiram, however, is more effective in those individuals motivated to stop drinking and when administration is supervised. Id. Naltrexone also has shortcomings, such as its side effect profile. Possible side effects of naltrexone at the 50 mg dose include heptatoxicty for those individuals who are also taking non-steroidal anti-inflammatory drugs (NSAIDs) and gastrointestinal and central nervous problems. Id.

Medications that further reduce the powerful craving of alcohol are nalmefene and ondansetron. According to nalmefene's package insert, the most common side effects when administered at higher doses, include nausea, vomiting, tachycardia and hypertension. Ondansetron has not been approved by the FDA for the treatment of alcoholism. Side effects for ondansetron may include irregular heartbeat, muscle cramps, headache, fatigue, anxiety and diarrhea.

Alcohol-related damage to the mind and brain encompasses many severe diseases, disorders or conditions. For instance, alcohol-related damage to the mind and brain can cause cognitive difficulty and abnormal behavior. The resulting abnormal behavior includes, but is not limited to, agitation, aggression, dyskinesis and hyperkinesis. Cognitive difficulty from alcohol intake includes cognitive problems due to alcohol intake and alcohol-mediated disorders of cognition.

There is also evidence that the frontal lobes are susceptible to damage from alcohol consumption. Oscar-Berman, M. and Marinkovic, K., Alcohol Research & Health 27(2): 125-132 (2003). The prefrontal cortex, which is the anterior region of the frontal lobes, controls the normal functioning of cognitive, emotional and interpersonal behaviors. Id. Examples of prefrontal controlled behavior are goal directed behaviors, good judgment and problem-solving skills. Id. Further, the left hemisphere, which is largely responsible for communication and the understanding of both written and spoken words, and the right hemisphere, which is involved in spatial cognition, are also affected by alcohol intake. Id.

Treatment for the behavioral effects of alcohol and alcohol-induced cognitive problems are minimal. The most effective way to prevent behavioral and cognitive complications of alcohol intake is abstinence. The effect of abstinence, however, will vary on the individual. In some alcoholics, for example, abstinence from alcohol may slowly reverse cognitive damage, while in other individuals the cognitive damage is irreversible. Oscar-Berman, M. et al., Alcohol Health & Research World 21(1): 65-75 (1997). Pharmaceutical treatments have been developed to treat alcohol-induced cognitive problems; however, none have been completely successful. Id. Additionally, sudden abstinence from alcohol may result in withdrawal symptoms described above.

Alcohol intake can also lead to alcohol-induced psychotic disorders. Principal treatment approaches for psychotic disorders include use of anti-psychotic medications. There is evidence that anti-psychotic medications, particularly the newer medications, may be useful in treating both alcohol misuse and the induced psychotic symptoms. Petrakis, I. L. et al. National Institute on Alcohol Abuse and Alcoholism Publications, Comorbidity of Alcoholism and Psychiatric Disorder—An Overview (2002). Side effects associated with anti-psychotic drugs include, for example, cognitive difficulties, sedation, orthostatic hypotension, changes in heart rate and rhythm, incontinence, and reduced appetite.

Altered sleep stages as a result of alcohol intake has affected the lives of many individuals. For example, insomnia affects 36 to 72 percent of alcoholic patients. Brower, K. J., Sleep Med Rev, 7(6): 523-39 (2003). Individuals who have altered sleep patterns also experience feelings of depression and anxiety, which may result in increased alcohol consumption in an effort to fall asleep. Id. Treatment of altered sleep stages as a result of alcohol intake is limited. Although abstinence is an option, abstinence is often unsuccessful because altered sleep patterns may persist for months after one has ceased alcohol intake. Id.

Cerebellar degeneration is also a serious consequence of chronic, long-term alcohol intake. The cerebellum is responsible for muscle coordination, sensory and cognitive functions. Oscar-Berman, M. and Marinkovic, K., Alcohol Research & Health 27(2): 125-133 (2003). Those with cerebellar degeneration will experience movement difficulty. Id. There are no known medicinal therapies for the prevention, treatment and reversal of cerebellar degeneration. The only method available for treatment is abstinence.

Wernicke's encephalopathy is commonly associated with chronic alcohol use. Robinson, K., Emergency Nurse 11(5): 30-33 (2003). Wernicke's encephalopathy is a serious condition caused by a thiamine deficiency. Robinson, K., Emergency Nurse 11(5): 30-33 (2003). Thiamine deficiency is severe in chronic alcohol users because of general malnourishment experienced by alcoholics and because alcohol inhibits thiamine absorption. Robinson, K., Emergency Nurse 11(5): 30-33 (2003). Those who have Wernicke's encephalopathy suffer severe symptoms, including confusion and problems with memory, language and reasoning. Oscar-Berman, M., and Marinkovic, K., Alcohol Research & Health 27(2): 125-133 (2003). Wernicke's encephalopathy, if untreated, will lead to irreversible brain damage. Robinson, K., Emergency Nurse 11(5): 30-33 (2003). Death is the outcome in 17 to 20 percent of Wernicke's cases. Id. There is evidence that the other 85 percent of Wernicke's cases develop Korsakoff's syndrome, a type of psychosis. Id. Korsakoff's syndrome is characterized by memory and behavioral complications. Oscar-Berman, M. and Marinkovic, K., Alcohol Research & Health 27(2): 125-133 (2003).

Treatments for both Wernicke's and Korsakoff's syndrome are similar and equally ineffective. A common treatment approach focuses on restoring thiamine, for example, by the restoration of thiamine through administration of oral B vitamins. However, oral B vitamin administration is ineffective if the individual continues to consume alcohol because alcohol is a cause of poor absorption. Robinson, K., Emergency Nurse 11(5): 30-33 (2003). A second method for restoring thiamine is by injection. Injections are also ineffective because patients are not always willing to attend injection appointments. Id. Another treatment option for some patients is hospitalization. Id. Hospitalization, however, is very expensive.

The pituitary is also negatively affected by chronic alcohol intake and can lead to alcohol-induced hypopituitarism. Alcohol-induced hypopituitarism causes the pituitary to shrink, which leads to decreased output. Eventually, alcohol-induced hypopituitarism results in the functional failure of the pituitary. The only treatment available for alcohol-induced hypopituitarism is abstinence.

The peripheral nervous system is also affected by alcohol intake. Alcoholic peripheral neuropathy is a peripheral nervous system disorder characterized by numbness and weakness in the hands and feet. Oscar-Berman, M. et al., Alcohol Health & Research World 21(1): 65-75 (1997).

Mood disorders including, for example, those that are alcohol-induced, affect millions of people each year. Depression and anxiety affected approximately 19 million Americans in 1999. National Mental Health Association, Depression—What You Need to Know Factsheet (1999). Further, mood disorders affect over 13 percent of all alcoholics. Kranzler, H., Rosenthal, R., The American Journal on Addictions 12, S26-S40 (2003).

Tricyclic antidepressants have been thought to reduce depressive symptoms and anxiety in alcoholics and non-alcoholics. Id. The side effect profile of tricyclic antidepressants can be mild to severe, including mild tremors, weakness, constipation and the most severe being death if taken at high doses.

Selective serotonin reuptake inhibitors (SSRIs) are also used to reduce depressive symptoms and anxiety in alcoholics and non-alcoholics. Although SSRIs are reported to have a better safety profile than tricyclic antidepressants, in that death is not caused when taken at high doses, SSRI side effects include nausea, loss of appetite, diarrhea, headache, and decreased sex drive. The FDA has also advised that certain patients on SSRI medication should be closely monitored for warning signs of suicide.

Anticonvulsant treatment and cognitive behavioral therapy for depression may also be used to reduce depressive symptoms. Id. The side effect profile for anticonvulsants includes drowsiness, restlessness, irritability, confusion, dizziness, nausea and fever.

In addition to tricyclic antidepressants and SSRIs, medications available for treating alcohol-induced anxiety and other mood disorders include benzodiazepines. Petrakis, I. L. et al., National Institute on Alcohol Abuse and Alcoholism Publications, Comorbidity of Alcoholism and Psychiatric Disorder—An Overview (2002). Although benzodiazepines are used for treating alcohol-induced anxiety, individuals may have a tendency to abuse benzodiazepines.

Hallucinations and paranoid delusions may also be caused by alcohol consumption. Beers, M. H. and Berkow, R., The Merck Manual of Diagnosis and Therapy, 17th Ed., 1593-1594; 1564-1571 (1999). Delusions are characterized by false beliefs. Id. Hallucinations are false sensory perceptions and may be auditory, visual, olfactory, gustatory or tactile. Id. Treatment of hallucinations and paranoid delusions will depend on the severity and cause. Treatment of paranoid delusions and auditory and visual hallucinations caused by amphetamine dependence or schizophrenia, for example, may involve phenothiazines, including chlorpromazine and haloperidol. Id. Chlorpromazine 25 to 50 mg IM can reverse paranoid delusions and visual and auditory hallucinations; however, it may cause serious postural hypotension. Although haloperidol is unlikely to cause hypotension, it may cause acute extrapyramidal motor reaction. Id.

Alcohol consumption also has detrimental effects on the digestive system, and may lead to esophagitis, acute gastritis, chronic gastritis, acute pancreatitis and chronic pancreatitis.

Esophagitis is inflammation of the esophagus, which occurs as a result of being irritated by alcohol intake. Bode, C., Bode, J. C., Alcohol Heath & Research World 21(1): 76-83 (1997). Esophagitis treatments, for the most part, decrease the symptoms caused by esophagitis. These treatments include the administration of acid blocking medication, pain and corticosteroid medications. For more severe damage, surgical removal of damaged tissue of the esophagus may be necessary.

Acute and chronic gastritis is inflammation of the stomach lining, more specifically known as mucosal inflammation. Acute gastritis is short-term and can be reversed. Id. Chronic gastritis, however, results from chronic alcohol exposure and leads to permanent damage of the mucosa. Id. Both acute and chronic gastritis may lead to many uncomfortable symptoms such as abdominal pain, nausea, vomiting and flatulence. Id. Treatments for acute and chronic gastritis include medications that reduce stomach acid, which unfortunately can cause additional irritation to inflamed areas, and avoidance of spicy foods.

Alcohol consumption affects the pancreas by causing acute pancreatitis. With continual chronic alcohol consumption, acute pancreatitis will develop into chronic pancreatitis. Apte, M. V. and Wilson, J. S., Alcohol Health & Research World 21(1): 13-20 (1997). In either form, alcohol-induced pancreatitis may destroy all parts of the pancreas and can be fatal. Id. Hundreds of people die every year from alcoholic pancreatitis. Common symptoms include severe abdominal pain and abnormal pancreatic functioning. Id. Treatment for acute and chronic pancreatitis needs to be improved. For both conditions, abstinence from alcohol may lesson abdominal pain and may slow the progression of pancreatitis. Id. Additional treatment for acute pancreatitis includes bed rest, appropriate pain relief, fasting and intravenous fluids. Id. Other than pain relief, additional treatment for chronic pancreatitis depends on the resulting damage. If a patient experiences abnormal pancreatic enzyme excretion, usually pancreatic enzymes will be administered. Where the patient has become diabetic, insulin or hypoglycemic agents are administered. Id.

It has been reported that alcoholic cardiomyopathy may develop in individuals who consume alcohol. Alcoholic cardiomyopathy is identified by low cardiac output and enlargement of the heart. Zakhari, S., Alcohol Health & Research World 21(1): 21-39 (1997). A severe effect of alcoholic cardiomyopathy is congestive heart failure, which may lead to death. Id. Hundreds of Americans die each year due to alcoholic cardiomyopathy. Center for Disease Control and Prevention, Alcohol-Attributable Deaths Report-United States 2001 (2001).

The main treatment for alcoholic cardiomyopathy is abstention to improve heart function. Zakhari, S., Alcohol Health & Research World 21(1): 21-39 (1997). After abstention from alcohol, alcoholic cardiomyopathy may be reversible; however, more severe cases may still progress to congestive heart failure regardless of alcohol intake. Id. Because alcoholic cardiomyopathy can lead to congestive heart failure, the standard treatment for heart failure may also be administered. Further, hospitalization may be necessary depending on the severity and alcohol dependence.

Cardiac rhythm can also be negatively affected by alcohol intake, which leads to alcohol-induced cardiac arrhythmias. Both acute and chronic alcohol consumption are reported to cause cardiac arrhythmias. Id. Alcohol-induced cardiac arrhythmias have serious consequences, the most severe being death. Id. Treatment for alcohol-induced cardiac arrhythmias depends on the severity of the condition. Medication will be prescribed when necessary, but the potent medications used for arrhythmias have many adverse side effects. As with many heart conditions, electronic shock therapy, pacemakers, and open heart surgery may be necessary for patients.

Skeletal muscle is vulnerable to alcohol-related damage resulting in the condition known as alcoholic myopathy. Alcoholic myopathy is widespread and affects 40 to 60 percent of chronic alcoholics. Adachi, J. et al., Journal of Nutritional Biochemistry 14(11): 616-25 (2003). Alcoholic myopathy is caused by the atrophy of Type II muscle fibers. Id. Alcoholic myopathy symptoms include muscle cramps, decreased muscle mass, and strength. Id. The only treatment available for alcoholic myopathy is abstinence.

Alcohol consumption has also been described as a risk factor for infection with Mycobacterium tuberculosis. Tuberculosis is caused by the inhalation of Mycobacterium tuberculosis bacteria into the lungs. Although the infection normally remains in the lungs, which results in a condition known as pulmonary tuberculosis, the bacteria may travel to other parts of the body resulting in a condition known as extrapulmonary tuberculosis, which includes tuberculosis meningitis, tuberculosis pericarditis and renal tuberculosis. An individual with a healthy immune system will be able to fight off the infection, however, those with a weaker immune system risk developing extrapulmonary tuberculosis.

Tuberculosis has affected approximately 32% of the world's population. Smith, K. C. et al., Expert Review of Anti-infective Therapy 1(3): 483-91 (2003). It was reported that an estimated two million people die annually from tuberculosis. Id. Common symptoms of active tuberculosis include coughing up thick, cloudy, and possibly bloody mucus for a few weeks; rapid heartbeat; swelling in the neck; and back pain. Due to drug resistant tuberculosis, treatment of tuberculosis involves a multi-drug approach. Antibiotics such as rifampin, isoniazid, pyrazinamid and ethambutaol are available for treatment, however, all have side effects. Common side effects of rifampin, isoniazid and pyrazinamid include nausea, diarrhea, painful urination, worsening gout, seizures, blurred vision and abnormal behavior. Common side effects of ethambutol include rash, vision changes, and hallucinations. Surgery may be necessary to repair tissue damage caused by tuberculosis or to remove a pocket of bacteria.

There is a clear need for improved methods to treat the foregoing diseases, conditions or disorders. The methods of the present invention described and claimed herein provide such improved methods.

### BRIEF SUMMARY

The inventions described and claimed herein have many attributes and embodiments including, but not limited to, those set forth or described or referenced in this Brief Summary. The inventions described and claimed herein may not be restricted to, or limited to or by, the features or embodiments identified in this Brief Summary.

The present invention is directed, in part, to methods of treatment including, for example, methods of treating various alcohol-related diseases, disorders, and/or conditions, by administration of a composition comprising a therapeutically effective amount of a glyponectin, a glyponectin agonist, an adiponectin and/or an adiponectin agonist to a mammal in need thereof.

In certain embodiments, the invention provides methods of treating alcohol-related diseases, disorders and/or conditions of the mind and/or brain, comprising administration of a composition comprising a therapeutically effective amount of a glyponectin, a glyponectin agonist, an adiponectin and/or an adiponectin agonist to a mammal in need thereof. In another aspect, the invention provides, for example, a method of treating diseases, disorders and/or conditions having any of the characteristics of alcohol-related diseases, disorders and/or conditions of the mind and/or brain, comprising administering a composition comprising a therapeutically effective amount of a glyponectin, a glyponectin agonist, an adiponectin and/or an adiponectin agonist to a mammal in need thereof.

The alcohol-related diseases, disorders or conditions of the mind and/or brain may include, for example, any one or more of the diseases, disorders or conditions relating to alcohol intake, excessive alcohol intake, chronic alcohol intake and/or chronic excessive alcohol intake.

In one aspect of the invention, the alcohol-related diseases, disorders or conditions of the mind and/or brain include behavioral effects, for example, any one or more of agitation, aggression, dyskinesis or hyperkinesis.

The alcohol-related diseases, disorders or conditions of the mind and/or brain may also be, for example, any one or more of cognitive problems due to alcohol intake. Cognitive problems may include, for example, impaired memory function, deficits in problem solving and complex thinking.

The alcohol-related diseases, disorders or conditions of the mind and/or brain may be, for example, any one or more of Wernicke's syndrome, Korsakoff's syndrome, alcoholic cerebellar degeneration or alcohol-induced hypopituitarism.

In another aspect of the invention, the alcohol-related diseases, disorders or conditions of the mind and/or brain may also be altered sleep stages as a result of alcohol intake, including, but not limited to insomnia, delayed sleep onset, frequent awakenings, and reduced amounts of nonREM sleep, sleep apnea and hypoxia.

In another aspect of the invention, the alcohol-related diseases, disorders or conditions of the mind and/or brain may also be alcoholic peripheral neuropathy.

In yet another aspect of the invention, the alcohol-related diseases, disorders or conditions of the mind and/or brain may also be alcohol-induced psychotic disorder.

In a further aspect, the present invention includes, for example, a method of treating hallucinations comprising, administering a composition comprising a therapeutically effective amount of a glyponectin, a glyponectin agonist, an adiponectin, and/or an adiponectin agonist to a mammal in need thereof. The hallucinations may include, for example, hallucinations that are alcohol-induced.

In a further aspect the present invention includes, for example, a method of treating paranoid delusions, comprising administering a therapeutically effective amount of a glyponectin, a glyponectin agonist, an adiponectin, and/or an adiponectin agonist to a mammal in need thereof. The paranoid delusions may include, for example, paranoid delusions that are alcohol-induced.

In a further aspect the present invention includes, for example, a method of treating mood disorders, comprising administering a therapeutically effective amount of a glyponectin, a glyponectin agonist, an adiponectin, and/or an adiponectin agonist to a mammal in need thereof. The mood disorders may include, for example, mood disorders that are alcohol-induced. In one aspect of the invention, mood disorders may include, for example, depression, sadness, and anxiety.

In a further aspect the present invention provides, for example, a method of treating increased levels and/or patterns of alcohol consumption and/or diseases, disorders or conditions having any of the characteristics of increased levels and/or patterns of alcohol consumption which comprises administering a therapeutically effective amount of a glyponectin, a glyponectin agonist, an adiponectin, and/or an adiponectin agonist to a mammal in need thereof.

Diseases, disorders or conditions having any of the characteristics of increased levels and/or patterns of alcohol consumption, may include, for example, any one or more of acute alcohol intoxication, chronic alcohol intoxication, alcohol abuse, alcoholism or alcohol dependence.

In still a further aspect, the invention includes, for example, a method of treating alcohol withdrawal syndrome, and/or diseases, disorders and conditions having any of the characteristics of alcohol withdrawal syndrome, comprising administering a therapeutically effective amount of glyponectin, a glyponectin agonist, an adiponectin, and/or an adiponectin agonist to a mammal in need thereof. Alcohol withdrawal syndrome may include, for example, irritability, agitation, tremors, delirium tremens, and seizures.

In yet another aspect, the present invention includes, for example, a method for treating alcohol-induced digestive system diseases, disorders or conditions and/or diseases, disorders or conditions having any of the characteristics of alcohol-induced digestive system diseases, disorders or conditions, comprising administering a therapeutically effective amount of a glyponectin, a glyponectin agonist, an adiponectin, and/or an adiponectin agonist to a mammal in need thereof. The digestive system diseases, disorders or conditions may be, for example, any one or more of esophagitis, acute gastritis, chronic gastritis, acute pancreatitis or chronic pancreatitis.

The present invention further provides, for example, a method of treating alcohol-induced cardiovascular diseases, disorders or conditions and/or diseases, disorders or conditions having any of the characteristics of alcohol-induced cardiovascular diseases, disorders or conditions, comprising administering a therapeutically effective amount of a glyponectin, a glyponectin agonist, an adiponectin, and/or an adiponectin agonist to a mammal in need thereof. The alcohol-induced cardiovascular diseases, disorders or conditions may be, for example, any one or more of alcoholic cardiomyopathy, alcohol-induced cardiac arrhythmias, hypertension, coronary artery disease, and stroke.

In still another aspect, the invention provides, for example, a method of treating alcoholic myopathy and/or diseases, disorders and conditions having any of the characteristics of alcoholic myopathy, comprising administering a therapeutically effective amount of a glyponectin, a glyponectin agonist, an adiponectin, and/or an adiponectin agonist to a mammal in need thereof.

In another aspect, the invention provides, for example, a method of treating extrapulmonary tuberculosis and/or disease, disorders and conditions having any of the characteristics of extrapulmonary tuberculosis comprising administering a therapeutically effective amount of glyponectin, a glyponectin agonist, an adiponectin, and/or an adiponectin agonist to a mammal in need thereof.

In a preferred embodiment of the invention, the glyponectin and/or adiponectin is recombinant. In another embodiment, the glyponectin and/or adiponectin is isolated, purified, or synthesized. In another preferred embodiment the glyponectin and/or adiponectin is human glyponectin and/or adiponectin. In yet another preferred embodiment the glyponectin is recombinant, human glyponectin. In yet another preferred embodiment the adiponectin is recombinant, human adiponectin. In another preferred embodiment the mammal is a human.

In some embodiments, the glyponectin is about 50% pure. In other embodiments, the glyponectin is at least about 70% pure, at least about 75% pure, at least about 80% pure, or at least about 85% pure. Preferably, the glyponectin is at least about 90% pure. More preferably the glyponectin is at least about 95% pure, at least about 97%, at least about 98% pure and most preferably the glyponectin is at least about 99% pure.

In some embodiments, the adiponectin is about 50% pure. In other embodiments, the adiponectin is at least about 70% pure, at least about 75% pure, at least about 80% pure, or at least about 85% pure. Preferably, the adiponectin is at least about 90% pure. More preferably the adiponectin is at least about 95% pure, at least about 97%, at least about 98% pure and most preferably the adiponectin is at least about 99% pure.

Still more preferably, an adiponectin is glycosylated (glyponectin). Glycosylated adiponectin may include, for example, (1) a glyponectin or glyponectin agonist wherein at least one of the lysine residues corresponding to lysine residues 65, 68, 77, and 101 of human adiponectin is glycosylated; (2) a glyponectin or glyponectin agonist wherein at least two of the lysine residues corresponding to lysine residues 65, 68, 77, and 101 of human adiponectin is glycosylated; (3) a glyponectin or glyponectin agonist wherein at least three of the lysine residues corresponding to lysine residues 65, 68, 77, and 101 of human adiponectin are glycosylated; (4) a glyponectin or glyponectin agonist wherein all four of the lysine residues corresponding to lysine residues 65, 68, 77, and 101 of human adiponectin are glycosylated; (5) a glyponectin or glyponectin agonist as defined in any of (1)-(4) wherein the glycosylation is with, for example, any one or more of a glucosylgalactosyl moiety, a glucosylglucosyl moiety, a galactosylglucosyl moiety, or a galactosylgalactosyl moiety; (6) a glyponectin or glyponectin agonist wherein the adiponectin is glycosylated and wherein the glyponectin or glyponectin agonist is (in whole or in part) a recombinant protein or polypeptide, an isolated protein or polypeptide, a purified protein or polypeptide, and/or a synthesized protein or polypeptide; (7) a glyponectin or glyponectin agonist wherein one or more of the residues corresponding to lysine residues 65, 68, 77 and 101 of human adiponectin are, for example, α-1-2-glucosylgalactosyl-O-hydroxylysines (8) a glyponectin or glyponectin agonist wherein the residue corresponding to proline residue 91 of human glyponectin is not hydroxyproline; (10) a glyponectin or glyponectin agonist wherein the residue corresponding to proline residue 91 of human glyponectin is hydroxyproline; and, (10) a glyponectin agonist having a desired level of glyponectin activity as compared against a naturally occurring glyponectin.

The glyponectin, glyponectin agonist, adiponectin, and/or adiponectin agonist preparation for use in the methods of the invention may be formulated in a manner suitable for administration to a mammal, for example, a human. The glyponectin, glyponectin agonist, adiponectin, and/or adiponectin agonist of the invention may be prepared for administration via an oral, transdermal, transmucosal, parenteral, suppository, inhalation, sublingual, and/or implantable delivery system. Parenteral administration may include, for example, subcutaneous (s.c.), intradermal (i.d.), intravenous (i.v.), intraperitoneal (i.p.) or transdermal administration. Other preparations are also envisaged in which said glyponectin is administered via other routes known in the art.

### BRIEF DESCRIPTION OF THE DRAWINGS

FIG. 1 shows the separation of adipocyte secreted adiponectin by 2-dimensional electrophoresis (2-DE) and the multiple isoforms of adiponectin. 1A shows 2-DE analysis of proteins in pre-adipocyte 3T3-L1 cells, where no adiponectin is detected. 1B shows 2-DE analysis of differentiated 3T3-L1 adipocytes wherein 8 isoforms of adiponectin are seen.

FIG. 2 shows the detection of 6 glycoisoforms detected after 2-DE separation of 3T3-L1 adipocyte proteins.

FIG. 3 shows the MALDI-TOF mass spectra analysis of tryptic peptides B (3A) (SEQ ID NOS 13-15) and C (3B) (SEQ ID-NOS 13-15).

FIG. 4 shows the MALDI-TOF mass spectra analysis of (A) bacterially produced, recombinant adiponectin; (B) isoform 1 of adiponectin secreted from 3T3-L1 adipocytes; (C) glycoisoform 3 of adiponectin secreted from 3T3-L1 adipocytes; and (D) glycoisoform 3 of recombinant adiponectin expressed in COS-7 cells.

FIG. 5 compares the expression of recombinant adiponectin and adiponectin (K→R) wherein the lysines were changed to arganines using site-directed mutagenesis. 4A demonstrates that when these two proteins are run out on a SDS-PAGE gel, the recombinant adiponectin runs as three bands, while the mutated adiponectin (K→R) runs as a single band. 4B demonstrates that glycosylation is abolished in the mutated adiponectin (K→R).

FIG. 6 shows the results of the expression of radiolabeled adiponectin, expressed in COS-7 cells with either [1-³H]glycosyl or [1-³H]galactosyl. The graph demonstrates that the glycosides on the four hydroxylysines in recombinant glyponectin expressed in COS-7 contain glucosyl and galactosyl.

FIG. 7 shows a sequence comparison of adiponectin between various animals and the highly conserved lysine groups.

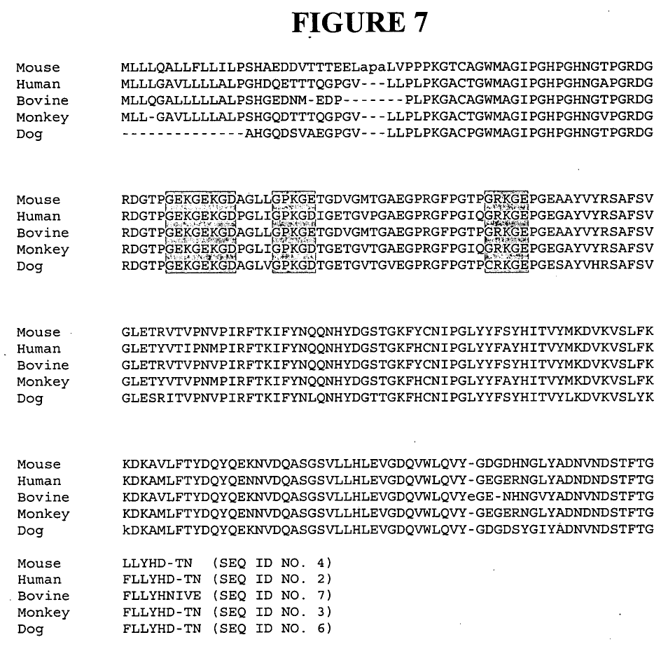

FIG. 8 demonstrates that the glycosylated lysine residues (SEQ ID NOS 16-18) in glyponectin are highly conserved across different species (SEQ ID NOS 19-23).

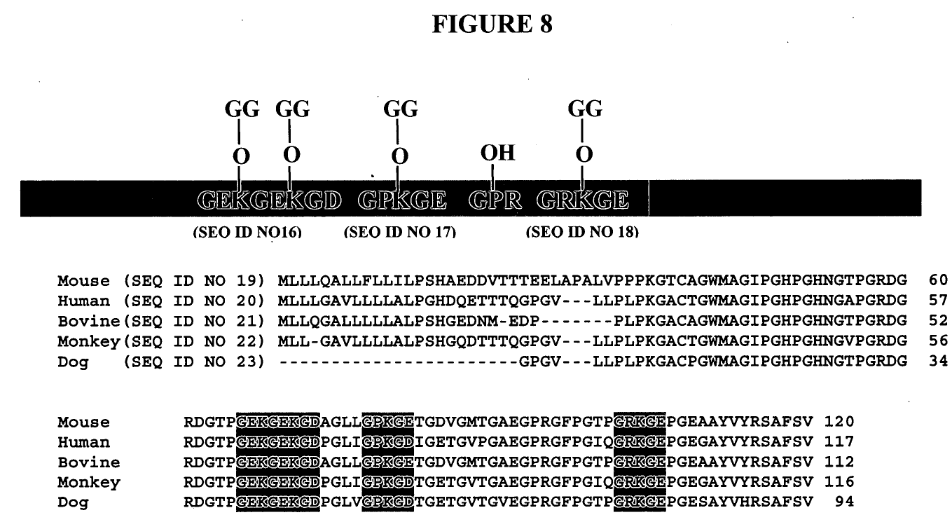

### DETAILED DESCRIPTION OF THE INVENTION

The practice of the present invention will employ, unless otherwise indicated, conventional techniques of molecular biology (including recombinant techniques), microbiology, cell biology, biochemistry, nucleic acid chemistry, and immunology, which are within the skill of the art. Such techniques are explained fully in the literature, such as, Molecular Cloning: A Laboratory Manual, second edition (Sambrook et al., 1989) and Molecular Cloning: A Laboratory Manual, third edition (Sambrook and Russel, 2001), (jointly referred to herein as “Sambrook”); Current Protocols in Molecular Biology (F. M. Ausubel et al., eds., 1987, including supplements through 2001); PCR: The Polymerase Chain Reaction, (Mullis et al., eds., 1994); Harlow and Lane (1988) Antibodies, A Laboratory Manual, Cold Spring Harbor Publications, New York, and Harlow and Lane (1999) Using Antibodies: A Laboratory Manual, Cold Spring Harbor Laboratory Press, Cold Spring Harbor, N.Y. (jointly referred to herein as “Harlow and Lane”), Beaucage et al. eds., Current Protocols in Nucleic Acid Chemistry, John Wiley & Sons, Inc., New York, 2000).

As used herein, the term “glyponectin” refers to recombinant, isolated, purified, or synthetic glycosylated adiponectins. Glyponectins include, for example, lysine glycosylated polypeptides as described herein.

Glyponectin and adiponectin nucleic acid or polynucleotide constructs refer to nucleic acid or polynucleotide constructs comprising a sequence coding for an adiponectin that is capable of being glycosylated.

“Substantially homologous” or “substantially similar” refers to sequence homology wherein at least about 50% of the sequences are identical, preferably at least about 60%, preferably at least about 65%, preferably at least about 70%, preferably at least about 75%, preferably at least about 80%, preferably at least about 90%, preferably at least about 95%, and more preferably at least about 99% nucleotide or amino acid residue identity, when compared and aligned for maximum correspondence. Sequences may include nucleic acid or amino acid sequences.

The terms “substantially pure” or “isolated,” refer to glyponectins and/or adiponectins of the invention that are separated as desired from RNA, DNA, proteins or other contaminants with which they are naturally associated. For example, when referring to proteins and polypeptides, a protein or polypeptide is considered substantially pure when that protein makes up greater than about 50% of the total protein content of the composition containing that protein, and typically, greater than about 60% of the total protein content. More typically, a substantially pure or isolated protein or polypeptide will make up at least about 75%, at least about 80%, at least about 85%, more preferably, at least about 90%, at least about 95% of the total protein. Preferably, the protein will make up greater than about 90%, and more preferably, greater than about 95% of the total protein in the composition.

The term “glyponectin agonist” refers to a molecule other than native glyponectin that exhibits at least one biological activity of native glyponectin. The term may include any molecule exhibiting such activity, and may be a polypeptide, polynucleotide, oligonucleotide, small molecule, organic molecule, inorganic molecule, a biomaterial, a carbohydrate, lipid, etc.

The term “adiponectin agonist” refers to a molecule other than native adiponectin that exhibits at least one biological activity of native adiponectin. The term may include any molecule exhibiting such activity, and may be a polypeptide, polynucleotide, oligonucleotide, small molecule, organic molecule, inorganic molecule, a biomaterial, a carbohydrate, lipid, etc.

As used herein, a “therapeutically effective amount” in reference to the compounds or compositions of the instant invention refers to the amount sufficient to induce a desired biological, pharmaceutical, or therapeutic result. That result can be alleviation of the signs, symptoms, or causes of a disease or disorder or condition, or any other desired alteration of a biological system. A therapeutically effective amount can be administered in one or more administrations by various routes of administration.

As used herein, the term “treating” refers to both therapeutic treatment and prophylactic or preventative measures. Those in need of treatment include those already with the disorder as well as those prone to having the disorder or diagnosed with the disorder or those in which the disorder is to be prevented. A treatment plan may occur over a period of time and may involve multiple dosages, multiple administrations, and/or different routes of administration.

As used herein, “preventing” means preventing in whole or in part, or ameliorating or controlling.

As used herein, “mammal” refers to any animal classified as a mammal, including humans, domestic and farm animals, and zoo, sports, or pet animals, such as dogs, horses, cats, sheep, pigs, cows, etc. The preferred mammal herein is a human.

As used herein the term “dosage forms” includes any appropriate dosage form well known in the art to be suitable for pharmaceutical formulation of polynucleotides or polypeptides suitable for administration to mammals, and in particular to humans, particularly (although not solely) those suitable for stabilization in solution of therapeutic polynucleotides or polypeptides for administration to mammals preferably humans. All this is irrespective of whether or not the glyponectin and/or adiponectin is in the form of a composition.

The present invention provides for administration of a composition comprising a glyponectin, a glyponectin agonist, an adiponectin, and/or an adiponectin agonist to a mammal useful to treat one or more disease states associated with alcohol-related diseases, disorders or conditions; including, for example, diseases, disorders or conditions of the mind and/or brain related to alcohol consumption; diseases, disorders or conditions of the mind and/or brain related to excessive alcohol consumption; diseases, disorders or conditions of the mind and/or brain related to chronic alcohol consumption; diseases, disorders or conditions of the mind and/or brain related to chronic, excessive alcohol consumption; hallucinations; alcohol-related hallucinations; paranoid delusions; alcohol-related paranoid delusions; mood disorders, including, for example, depression, sadness, and anxiety; alcohol-related mood disorders; increased levels and/or patterns of alcohol consumption; diseases, disorders or conditions having one or more characteristics of increased levels and/or patterns of alcohol consumption, including for example, alcohol intoxication, acute alcohol intoxication, chronic alcohol intoxication, alcohol abuse, alcoholism, and alcohol dependence; alcohol withdrawal syndrome; diseases, disorders or conditions having one or more characteristics of increased levels and/or patterns of alcohol withdrawal syndrome, including, for example, irritability, agitation, tremors, delirium tremens and seizures; alcohol-induced digestive system diseases, disorders or conditions, including, for example, esophagitis, acute gastritis, chronic gastritis, acute pancreatitis, and chronic pancreatitis; alcohol-induced cardiovascular diseases, disorders or conditions, including, for example, alcoholic cardiomyopathy, alcohol-induced cardiac arrhythmias, hypertension, coronary artery disease and stroke; alcohol-related cognitive problems, including for example, impaired memory function, deficits in problem solving and deficits in complex thinking; altered sleep stages related to alcohol intake, including, for example, insomnia, delayed sleep onset, frequent awakenings, reduced amounts of nonREM sleep, sleep apnea and hypoxia; alcoholic peripheral neuropathy; Wernicke's syndrome; Korsakoff's syndrome; alcoholic cerebellar degeneration; alcohol-induced hypopituitarism; alcoholic myopathy; and extra pulmonary tuberculosis.

In one embodiment, glyponectin and/or adiponectin has the sequence of a naturally occurring adiponectin, for example, from a mammal, including, for example, human (SEQ ID NO. 1 and 2), a non-human primate (SEQ ID NO. 3), a mouse (SEQ ID NO. 4), a rat (SEQ ID NO. 5), a dog (SEQ ID NO. 6), or a cow (SEQ ID NO. 7).

1. Human polynucleotide
(SEQ ID NO 1)
aggctgttga ggctgggcca tctcctcctc acttccattc tgactgcagt ctgtggttct
gattccatac cagaggggct caggatgctg ttgctgggag ctgttctact gctattagct
ctgcccggtc atgaccagga aaccacgact caagggcccg gagtcctgct tcccctgccc
aagggggcct gcacaggttg gatggcgggc atcccagggc atccgggcca taatggggcc
ccaggccgtg atggcagaga tggcacccct ggtgagaagg gtgagaaagg agatccaggt
cttattggtc ctaagggaga catcggtgaa accggagtac ccggggctga aggtccccga
ggctttccgg gaatccaagg caggaaagga gaacctggag aaggtgccta tgtataccgc
tcagcattca gtgtgggatt ggagacttac gttactatcc ccaacatgcc cattcgcttt
accaagatct tctacaatca gcaaaaccac tatgatggct ccactggtaa attccactgc
aacattcctg ggctgtacta ctttgcctac cacatcacag tctatatgaa ggatgtgaag
gtcagcctct tcaagaagga caaggctatg ctcttcacct atgatcagta ccaggaaaat
aatgtggacc aggcctccgg ctctgtgctc ctgcatctgg aggtgggcga ccaagtctgg
ctccaggtgt atggggaagg agagcgtaat ggactctatg ctgataatga caatgactcc
accttcacag gctttcttct ctaccatgac accaactga
2. Human polypeptide
(SEQ ID NO 2)
MLLLGAVLLLLALPGHDQETTTQGPGVLLPLPKGACTGWMAGIPGHPGHNGAPGRDGRDGTPGE
KGEKGDPGLIGPKGDIGETGVPGAEGPRGFPGIQGRKGEPGEGAYVYRSAFSVGLETYVTIPNM
PIRFTKIFYNQQNHYDGSTGKFHCNIPGLYYFAYHITVYMKDVKVSLFKKDKAMLFTYDQYQEN
NVDQASGSVLLHLEVGDQVWLQVYGEGERNGLYADNDNDSTFTGFLLYHDTN
3. Non-human primate
(SEQ ID NO. 3)
MLLGAVLLLLALPSHGQDTTTQGPGVLLPLPKGACTGWMAGIPGHPGHNGVPGRDGRDGTPGEK
GEKGDPGLIGPKGDTGETGVTGAEGPRGFPGIQGRKGEPGEGAYVYRSAFSVGLETYVTVPNMP
IRFTKIFYNQQNHYDGSTGKFHCNIPGLYYFAYHITVYMKDVKVSLFKKDKAMLFTYDQYQENN
VDQASGSVLLHLEVGDQVWLQVYGEGERNGLYADNDNDSTFTGFLLYHDTN
4. Mouse:
(SEQ ID NO 4)
MLLLQALLFLLILPSHAEDDVTTTEELAPALVPPPKGTCAGWMAGIPGHPGHNGTPGRDGRDGT
PGEKGEKGDAGLLGPKGETGDVGMTGAEGPRGFPGTPGRKGEPGEAAYMYRSAFSVGLETRVTV
PNVPIRFTKIFYNQQNHYDGSTGKFYCNIPGLYYFSYHITVYMKDVKVSLFKKDKAVLFTYDQY
QEKNVDQASGSVLLHLEVGDQVWLQVYGDGDHNGLYADNVNDSTFTGFLLYHDTN
5. Rat:
(SEQ ID NO 5)
MLLLQALLFLLILPSHEGITATEGPGALVPPPKETCAGWMAGIPGYPGHNGIPGRDGRDGTPGE
KGEKGDAGVLGPKGDPGDAGMTGAEGPRGFPGTPGRKGEPGEAAYMYHSAFSVGLETRVTVPNV
PIRFTKIFYNQQNHYDGSTGKFHCNIPGLYYFSYHITVYMKDVKVSLFKKDKAVLFTYDQYQEK
NVDQASGSMLLHLEVGDQVWLQVYGEGDNNGLYADNVNDSTFTGFLLYHDTN
6. Dog:
(SEQ ID NO 6)
MLLLRAVLLLLVLPAHGQDSVAEGPGVLLPLPKGACPGWMAGIPGHPGHNGTPGRDGRDGTPGE
KGEKGDAGLVGPKGDTGETGVTGVEGPRGFPGTPCRKGEPGESAYVHRSAFSVGLESRITVPNV
PIRFTKIFYNLQNHYDGTTGKFHCNIPGLYYFSYHITVYLKDVKVSLYKKDKAMLFTYDQYQEK
NVDQASGSVLLHLEVGDQVWLQVYGDGDSYGIYADNVNDSTFTGFLLYHDTN
7. Cow:
(SEQ ID NO 7)
MLLQGALLLLLALPSHGEDNMEDPPLPKGACAGWMAGIPGHPGHNGTPGRDGRDGTPGEKGEKG
DAGLLGPKGETGDVGMTGAEGPRGFPGTPGRKGEPGEAAYVYRSAFSVGLETRVTVPNVPIRFT
KIFYNQQNHYDGSTGKFYCNIPGLYYFSYHITVYMKDVKVSLFKKDKAVLFTYDQYQEKNVDQA
SGSVLLHLEVGDQVWLQVYEGENHNGVYADNVNDSTFTGFLLYHNIVE

The glyponectin and/or adiponectin may also have a sequence that is substantially similar to that of a naturally occurring adiponectin. Glyponectin and/or adiponectin may also be a mature form lacking the signal sequence of the pro- or the pre-pro form. Additionally, glyponectin and/or adiponectin may differ from a naturally occurring adiponectin by additions, deletions, substitutions conservative substitutions, derivitization and/or truncations (natural or recombinant),

Additions, deletions and substitutions may be created using methods known in art. For example, random or site-directed mutagenesis and other molecular biology techniques.

A conservative substitution in a protein is generally a substitution of one amino acid with an amino acid with similar size and charge. Groups of amino acids known normally to be equivalent are understood in the art and include, for example: (a) Ala, Ser, Thr, Pro, and Gly; (b) Asn, Asp, Glu, and Gln; (c) His, Arg, and Lys; (d) Met, Glu, Ile, and Val; and (e) Phe, Tyr, and Trp. In one embodiment, conservative substitutions are substantially homologous to native glyponectin and retain at least a part of its biological activity as disclosed herein.

Glyponectin and/or adiponectin may also include fragments of a full-length glyponectin and/or adiponectin, such as may be obtained by deletion of one or more amino acid residues of full-length glyponectin and/or adiponectin or truncation of full-length glyponectin and/or adiponectin. Active fragments or portions of glyponectin and/or adiponectin may be ascertained by stepwise deletions of amino acid residues, from the N-terminal end or the C-terminal end or from within the glyponectin and/or adiponectin peptide. Other methods for creating truncations or deletions are known in the art.

Glyponectin and/or adiponectin may also include fragments retaining, in whole or in part, one or more biological activities of glyponectin and/or adiponectin.

Biologically active fragments include, but are not limited to, fragments comprising amino acid residues corresponding to 82-244, 101-244, 110-244 of human adiponectin and/or 104-247 of mouse adiponectin.

Other fragments include, for example, amino acid residues 42-244 (see, e.g., PCT App. No. WO03055916), 58-244 (Id.), 84-244 (see, e.g., U.S. Pat. App. No. 2004/0067881), 85-244 (Id.), 86-244 (Id.), 87-244 (Id.), 88-244 (see, e.g., Id., and PCT App. No. WO03055916), 89-244 (see, e.g., U.S. Pat. App. No. 2004/0067881), 90-244 (Id.), 91-244 (Id.), 92-244 (Id.), 93-244 (Id.), 94-244 (Id.), 95-244 (Id.), 96-244 (Id.), 97-244 (Id.), 98-244 (Id.), 99-244 (see, e.g., Id., and PCT App. No. WO03059934), 100-244 (see, e.g., PCT App. No. WO03055916, and U.S. Pat. App. No. 2004/0067881), 102-244 (see, e.g., U.S. Pat. App. No. 2004/0067881), 103-244 (Id.), 105-244 (Id.), 108-244 (see, e.g., PCT App. No. WO03055916 and U.S. Pat. No. 6,579,852), and 111-191 (see, e.g., U.S. Pat. App. No. 2004/0067881), 115-244 (see, e.g., PCT App. No. WO03055916), 132-244 (see, e.g., U.S. Pat. No. 6,579,852 and PCT App. No. WO03055916), 144-199 (see, e.g., U.S. Pat. App. No. 2004/0067881), 166-193 (see, e.g., PCT App. No. WO03044057), 166-176 (Id.), 167-176 (Id.), 191-244 (see, e.g., U.S. Pat. App. No. 2004/0067881).

Other fragments include, but are not limited to, amino acid residues 88-247 (see, e.g., U.S. Pat. App. No. 2004/0067881), 89-247 (Id.), 90-247 (Id.), 91-247 (Id.), 92-247 (Id.), 93 -247 (Id.), 94-247 (Id.), 95-247 (Id.), 96-247 (Id.), 97-247 (Id.), 98-247 (Id), 100-247 (Id.), 101-247 (Id.), 102-247 (Id.), 103-247 (Id.), 105-247 (Id.), 106-247 )Id.), and 110-247 (see, e.g., U.S. Pat. App. No. 2003/0147855), 111-247 (see, e.g., U.S. Pat. No. 6,579,852) and 135-247 (Id.).

Other fragments are identified in U.S. Pat. App. No. 2004/0067881.

Preferably such fragments retain at least one glycosylation site found in full-length glyponectin, and may include at least two, at least three, or all such sites. Such fragments also may retain at least one hydroxyproline residue found in full-length glyponectin, and may include at least two, at least three or all such residues. The fragments may retain any or all of the biological activities exhibited by full-length glyponectin, including those known and described in the literature.

Active fragments or portions of glyponectin and/or adiponectin polypeptides may be fused with other polypeptides by methods well known in the art to yield a chimeric polypeptide. Any such chimeric polypeptides that retain the biological activity of native glyponectin and/or adiponectin are also considered to be glyponectin and/or adiponectin polypeptides of the invention.

Glyponectin and/or adiponectin may also include functional variants of naturally occurring glyponectin and/or adiponectin. A polynucleotide encoding a glyponectin and/or adiponectin may be subjected to any of various mutagenic or evolutionary methods to produce variants of the disclosed sequences. The variants may be selected to retain a biological activity of interest, and may be desirably altered in such activity. The selection process may occur after or during the generation of such variants. Chemical or biological mutagenesis methods can be used, including chemical treatment of isolated nucleotides or of host cells, site-directed mutagenesis techniques, random biological mutagenesis methods including replication under conditions of poor fidelity, and directed evolution techniques. Polynucleotides can be subjected to DNA shuffling techniques such as those developed by Stemmer (U.S. Pat. Nos. 5,605,793; 5,811,238; 5,830,721) using sequences described herein. Polynucleotides can be subjected to systematic cassette mutagenesis methods such as those described by Huse. Systematic analysis of all mutants at each position of the protein can also be used, and can be done using a series of position-specific degenerate nucleotides (Short, U.S. Pats. Nos. 6,054,267, 5,939,250, 5,763,239, 6,537,776, 6,238,884, 6,171,820, 5,830,696 5,965,408, and 5,955,358). Biased mutagenesis methods can be used, including the use of libraries based on substitution matrices (U.S. Pat. Publ. 2020155460 A1 published Oct. 24, 2002 to Schellenberger et al.). Combinations of such methods are also provided. Methods of producing such mutants are provided, as are methods of use of glyponectin described herein that incorporate such glyponectin variants.

Glyponectin and/or adiponectin may also include allelic variants, differential splice variants, alternative splice variations, and other naturally occurring variants having biological activity.

Also included within glyponectin and/or adiponectin fragments and variants are those polypeptides that are encoded by a nucleic acid sequence that hybridizes specifically under stringent hybridization conditions to the complementary strand to a nucleic acid sequence encoding a full length glyponectin described herein. “Stringent hybridization conditions” typically refers to conditions in a range from about 5° C. to about 20° C. or 25° C. below the melting temperature (Tm) of the target sequence and a probe with exact or nearly exact complementarity to the target. As used herein, the melting temperature is the temperature at which a population of double-stranded nucleic acid molecules becomes half- dissociated into single strands. Methods for calculating the Tm of nucleic acids are well known in the art (see, e.g., Berger and Kimmel, 1987, Methods In Enzymology, Vol. 152: Guide To Molecular Cloning Techniques, San Diego: Academic Press Inc., and Sambrook et al.; supra; (1989) Molecular Cloning: A Laboratory Manual, 2nd Ed., Vols. 1-3, Cold Spring Harbor Laboratory). As indicated by standard references, a simple estimate of the Tm value may be calculated by the equation: Tm=81.5+0.41(% G+C), when a nucleic acid is in aqueous solution at 1 M NaCl (see e.g., Anderson and Young, “Quantitative Filter Hybridization” in Nucleic Acid Hybridization (1985)). Other references include more sophisticated computations which take structural as well as sequence characteristics into account for the calculation of Tm. The Tm of a hybrid (and thus the conditions for stringent hybridization) is affected by various factors such as the length and nature (DNA, RNA, base composition) of the probe and nature of the target (DNA, RNA, base composition, present in solution or immobilized, and the like), and the concentration of salts and other components (e.g., the presence or absence of formamide, dextran sulfate, polyethylene glycol). The effects of these factors are well known and are discussed in standard references in the art (see, e.g., Sambrook, supra, and Ausubel, supra. Typically, stringent hybridization conditions are salt concentrations less than about 1.0 M sodium ion, typically about 0.01 to 1.0 M sodium ion at pH 7.0 to 8.3, and temperatures at least about 30° C. for short probes (e.g., 10 to 50 nucleotides) and at least about 60° C. for long probes (e.g., greater than 50 nucleotides). As noted, stringent conditions may also be achieved with the addition of destabilizing agents such as formamide, in which case lower temperatures may be employed.

Fragments, portions, variants comprising mutations, deletions and/or substitutions, functional variants, allelic variants, differential splice variants, and alternative splice variations of adiponectin and adiponectin agonists of the invention are also contemplated.

In one preferred embodiment, the glyponectin or glyponectin agonist of the invention is a human glyponectin or glyponectin agonist. In one preferred embodiment the glyponectin is glycosylated at one or more lysine residues corresponding to amino acid residues 65, 68, 77 or 101 of human adiponectin, if the glyponectin or glyponectin fragment includes one or more of said residues. In another preferred embodiment, at least two of the lysines corresponding to amino acid residues 65, 68, 77 or 101 of human adiponectin are glycosylated. In another preferred embodiment, at least three of the lysines corresponding to amino acid residues 65, 68, 77 or 101 of human adiponectin are glycosylated. In yet another preferred embodiment, all four of the lysines corresponding to amino acid residues 65, 68, 77 or 101 of human adiponectin are glycosylated. Additional glycosylation may be added and/or existing glycosylation sites moved as desired for biological activity.

Without wishing to be bound by any particular mechanism or theory, it is believed that the glycosylation at lysine residues is typically O-linked and can result in one or more sugar moieties being added to each lysine residue. In one embodiment of the invention, for example, the sugar moieties that are added to the lysine residues are one or more combinations of a glucosyl and/or galactosyl. For example, in a preferred embodiment the glyponectin polypeptide has at least one glucosylgalactosyl, glucosylglucosyl, galactosylgalactosyl or galactosylglucosyl moiety at one or more lysine residues, including lysine residues 68, 71, 80, and 104 (mouse) or residues 65, 68, 77, and 101 (human). In another embodiment, the glyponectin polypeptide is glycosylated by X,, at least one of lysine residues 68, 71, 80, and 104 (mouse) or residues 65, 68, 77, and 101 (human) or at all of Lys-68, 71, 80, and 104 (mouse) or Lys-65, 68, 77, and 101 (human) wherein each X is independently selected from one or more of a glucosylgalactosyl moiety, a glucosylglucosyl moiety, a galactosylgalactosyl moiety, and/or a galactosylglucosyl moiety. In one embodiment, all lysines in glyponectin polypeptides are glycosylated. In another embodiment of the invention, the sugar moiety that is added to the lysine residues is a α-1-2-glucosylgalactosyl-O-hydroxylysine.

In another embodiment of the invention, the glyponectin or glyponectin agonist comprises a hydroxyproline at the residue corresponding to proline residue 91 of human adiponectin. In another embodiment of the invention, the glyponectin or glyponectin agonist does not contain a hydroxyproline at the residue corresponding to proline residue 91 of human adiponectin.

Glyponectin polypeptides that differ from one another by the glycosylation, or lack thereof, for example, at one or more of the lysine residues including those corresponding to lysine residues 65, 68, 77 and 101 of human glyponectin, are sometimes referred to herein as “glycoisoforms.” Different isoforms and/or glycoisoforms can be distinguished on the basis of isoelectric point (pI) and apparent molecular weight. The different isoforms can be identified by standard methods, including electrophoresis. Glyponectin isolated from adipocytes exists in at least eight different isoforms, which can be defined according to pI and electrophoretic mobility (apparent molecular weight). Some isoforms are glycosylated (glycoisoforms) and others are not.

Whilst reference is made herein specifically to glycosylation by a sugar or mix of sugars or more specifically to various entities, including glucosylgalactosyl, glucosylglucosyl, galactosylgalactosyl and/or galactosylglucosyl moieties, the term includes within its scope any expansion or variation of that glycosylating moiety that elicits similar (although not necessarily quantitatively similar) biological activity to that more specifically identified.

Whilst reference is made herein specifically to hydroxylation the term includes within its compass any expansion or variation of that hydroxylating moiety including other modifications that elicits a similar biological activity to that more specifically identified.

Whilst reference is made herein specifically to hydroxyproline the term includes within its scope any amino acid including modified amino acids that elicits a similar biological activity to that more specifically identified.

As noted, the glyponectin or glyponectin agonist may be glycosylated with at least one sugar moiety at one or more lysine or other relevant residues within the glyponectin or glyponectin agonist. Mouse adiponectin lysine residues 68, 71, 80, and 104, and corresponding human adiponectin lysine residues 65, 68, 77, and 101, are targets for glycosylation (see, e.g., U.S. Pat. App. No. 2004/0023854).

In one aspect, the invention utilizes compositions containing, for example, human glycoisoforms, glycoisoforms from non-human species, and a glyponectin agonist polypeptide-glycosylated at one or more lysine residues, including one or more of the residues corresponding to lysine residues 65, 68, 77, and 101 of human glyponectin. It will be appreciated when referring to glyponectin of non-human species, a glyponectin variant, or a truncated glyponectin different from the human glyponectin, for example, the human glyponectin sequence shown in SEQ ID NO: 1 and 2, that the residues of the glyponectin can be referred to using the numbering corresponding to the human sequence residue, as determined by optimally aligning the two sequences. For example, in naturally occurring mouse glyponectin, four corresponding lysine residues are found at residues 68, 71, 80, and 104. It will be appreciated that, when discussing numbering in one species (e.g., human or mouse), the discussion is intended to refer also to the equivalent numbering in other species.

In one aspect, the invention utilizes a composition comprising a glyponectin or glyponectin agonist that is substantially free of at least one non-glycosylated isoform. In another aspect, the composition is substantially free of any non-glycosylated isoform. As used herein, a composition is “substantially free” from an isoform when that form is less than about 20%, preferably less than about 10%, preferably less than about 5%, most preferably less than about 1% or about 0.1% by weight of the glyponectin in the composition. Methods for obtaining such compositions include methods provided in U.S. Pat. App. No. 2004/0023854, as well as methods known in the protein purification and chromatography arts.

In yet another aspect, the invention utilizes a composition containing a glyponectin or glyponectin agonist wherein the only or the predominant adiponectin species is fully or partially glycosylated. In one embodiment, for example, the composition contains more than one isoform and/or glycoisoform in more than one glycosylation state. In this context, “predominant” refers to the composition in which at least about 50% of the glyponectin in the composition is in the specified glycosylation state or of the specified isoform, preferably at least about 60%, at least about 65%, at least about 70%, at least about 75%, at least about 80%, at least about 85%, at least about 90%, at least about 95%, at least about 98% and/or at least about 99%, glyponectin.

Several methods can be used to obtain a glyponectin and/or adiponectin of the invention. It will be appreciated that a glyponectin, a glyponectin agonist, an adiponectin and/or an adiponectin agonist can be produced by recombinant or synthetic means, or, as appropriate, isolated or purified from naturally occurring sources.

In one embodiment, one or more glyponectin, glyponectin agonist, adiponectin and/or adiponectin agonist are prepared by recombinant methods. In another embodiment one or more glyponectin, glyponectin agonist, adiponectin and/or adiponectin agonist of the invention are isolated from a mammal. In another embodiment, one or more glyponectin, glyponectin agonist, adiponectin and/or adiponectin agonist of the invention are prepared using polypeptide or polynucleotide synthesis methods known in the art.

Glyponectin, glyponectin agonist, adiponectin and/or adiponectin agonist polypeptides may be produced recombinantly by inserting a polynucleotide (usually DNA) sequence that encodes the protein into an expression vector and expressing the peptide in an appropriate host. A polynucleotide encoding the desired polypeptide, whether in fused or mature form, and whether or not containing a signal sequence to permit secretion, may be ligated into expression vectors suitable for any convenient host. Any of a variety of expression vectors known to those of ordinary skill in the art may be employed, however eukaryotic expression systems are recommended because of the ability of eukaryotic cells to perform post-translational modifications, such as glycosylation. Expression may be achieved in any appropriate host cell that has been transformed or transfected with an expression vector containing a DNA molecule that encodes the recombinant peptide(s). Examples of eukaryotic host cells are known in the art and include yeast, avian, insect, plant, and animal cells such as COS-1, COS7, HeLa, CHO, HEK-293 and other mammalian cells. Standard techniques for recombinant production are described, for example, in Sambrook, supra.

In another embodiment, a glyponectin, a glyponectin agonist, an adiponectin and/or an adiponectin agonist can be obtained by expression of a recombinant polynucleotide encoding adiponectin or a polypeptide having a sequence of any of SEQ ID NOS.: 1-7, for example, or a biologically active fragment, substitution, deletion or addition thereof, or other variant or adiponectin agonist, in mammalian cells.

In another embodiment, glyponectin and/or adiponectin (including mixtures of isoforms and glycoisoforms) can also be purified from an animal cells or tissue such as, but not limited to, serum or adipocytes. Methods useful for purifying glyponectin and/or adiponectin from adipocytes are well known in the art and further, for example, U.S. Pat. App. No. 2004/0023854. The animals from which the composition of glyponectin and/or adiponectin can be obtained include but are not limited to humans, mice, rats, dogs, bovines, and non-human primates.

Glyponectin and/or adiponectin polypeptides can be separated from other polypeptides by several methods known in the protein purification art. In one embodiment, for example, the separation is effected by two-dimensional electrophoresis and subsequent excision and elution of the protein from the gel. In another embodiment, the separation is effected by using an affinity column that selects on the basis of electrical charge. In another embodiment, the separation is effected by using an affinity column loaded with lectins. In other embodiments, alternate protein purification methods are used, e.g., immunoaffinity column, size-exclusion column, lectin affinity, hydrophobic interaction, reversed phase, anion and cation exchange chromatography, and the like (see, generally, R. Scopes, Protein Purification, Springer-Verlag, N.Y. (1982) and Deutscher, Methods in Enzymology Vol. 182: Guide to Protein Purification, Academic Press, Inc. N.Y. (1990)).

Glycoisoforms may be separated from non-glycosylated isoforms using a lectin column, for example, a concanavalin A or wheat germ agglutinin column, to bind glycoisoforms. Non-glycosylated isoforms will not bind to the column and thus, will flow through the column. The glycoisoforms are then eluted from the column to obtain a composition containing predominantly glycoisoforms.

Glycoisoforms, obtained either recombinantly or from animal tissues, can also be separated on the basis of molecular weight, pI, and/or the amount of glycosylation present within the glyponectin polypeptide by routine methods, e.g., electrophoresis or chromatography. In one embodiment, isoforms and/or glycoisoforms of glyponectin may be prepared by differential purification. For example, this involves obtaining a first composition containing at least two forms of glyponectin that differ in their degree or type of glycosylation and then separating the glyponectin forms based on the degree or type of glycosylation. This method produces a second composition that differs from the first composition in the glyponectin profile.

In another embodiment, various isoforms and/or glycoisoforms are obtained by running glyponectin, either obtained recombinantly or from animal tissues, on a two-dimensional gel, identifying the glycosylated species by an antibody, excising the spot or band of the glycoisoform, and eluting the glycoisoform from the band to obtain a substantially pure composition of one or more glycoisoform(s). It will be recognized that compositions of the invention can be made by routine techniques, such as those described above, and including separating and recombining specific isoforms and/or glycoisoforms to prepare desired embodiments.

The invention also is directed to doses, dosage forms, formulations, compositions and/or devices comprising one or more glyponectins, glyponectin agonists, adiponectins, and/or adiponectin agonists, including glycoisoforms and/or non-glycosylated isoforms, useful for therapy of diseases, disorders, and/or conditions in humans and other mammals and other disorders as disclosed herein. The use of these dosage forms, formulations compositions and/or devices of glyponectin, a glyponectin agonist, an adiponectin, and/or an adiponectin agonist, including glycoisoforms and/or non-glycosylated isoforms enables effective treatment of these conditions, through novel and improved formulations suitable for administration to humans and other mammals.

The invention provides, for example, dosage forms, formulations, devices and/or compositions containing one or more glyponectins, glyponectin agonists, adiponectins, and/or adiponectin agonists. The dosage forms, formulations, devices and/or compositions of the invention may be formulated to optimize bioavailability and to maintain plasma concentrations within a therapeutic range, including for extended periods, and results in an increase in the time that plasma concentrations of glyponectin, glyponectin agonists, adiponectins, and/or adiponectin agonists remain within a desired therapeutic range at the site or sites of action and/or within the systemic circulation of the subject. Controlled delivery preparations also optimize the drug concentration at the site of action and minimize periods of under and over medication, for example.

The dosage forms, formulations, devices and/or compositions of the invention may be formulated for periodic administration, including once daily administration, to provide low dose controlled and/or low dose long-lasting in vivo release of a glyponectin.

Glyponectins glyponectin agonists, adiponectins, adiponectin agonists, and other therapeutic molecules of the invention may be prepared for administration via oral, transdermal, transmucosal, parenteral, suppository, inhalation, sublingual, and/or implantable delivery systems. Parenteral administration may include, for example, subcutaneous (s.c.), intradermal (i.d.), intravenous (i.v.), intraperitoneal (i.p.) or transdermal administration. Other preparations are also envisaged in which said glyponectin is administered via other routes known in the art.

Examples of dosage forms suitable for oral administration include, but are not limited to tablets, capsules, lozenges, or like forms, or any liquid forms such as syrups, aqueous solutions, emulsions and the like, capable of providing a therapeutically effective amount of a glyponectin, glyponectin agonist, adiponectin, and/or adiponectin agonist, including glycoisoforms and/or non-glycosylated isoforms.

Examples of dosage forms suitable for transdermal administration include, but are not limited, to transdermal patches, transdermal bandages, and the like. Examples of dosage forms suitable for topical administration of the compounds and formulations of the invention are any lotion, stick, spray, ointment, paste, cream, gel, etc. whether applied directly to the skin or via an intermediary such as a pad, patch or the like.

Examples of dosage forms suitable for suppository administration of the compounds and formulations of the invention include any solid dosage form inserted into a bodily orifice particularly those inserted rectally, vaginally and urethrally.

Examples of dosage forms suitable for transmucosal delivery of the compounds and formulations of the invention include depositories solutions for enemas, pessaries, tampons, creams, gels, pastes, foams, nebulised solutions, powders and similar formulations containing in addition to the active ingredients such carriers as are known in the art to be appropriate.

Examples of dosage of forms suitable for injection of the compounds and formulations of the invention include delivery via bolus such as single or multiple administrations by intravenous injection, subcutaneous, subdermal, and intramuscular administration or oral administration.

Examples of dosage forms suitable for depot administration of the compounds and formulations of the invention include pellets or small cylinders of active agent or solid forms wherein the active agent is entrapped in a matrix of biodegradable polymers, microemulsions, liposomes or is microencapsulated.

Examples of infusion devices for compounds and formulations of the invention include infusion pumps containing one or more glyponectin, a glyponectin agonist, an adiponectin, and/or an adiponectin agonist, including glycoisoforms and/or non-glycosylated isoforms, at a desired amount for a desired number of doses or steady state administration, and include implantable drug pumps.

Examples of implantable infusion devices for compounds, and formulations of the invention include any solid form in which the active agent is encapsulated within or dispersed throughout a biodegradable polymer or synthetic, polymer such as silicone, silicone rubber, silastic or similar polymer.

Examples of dosage forms suitable for inhalation or insufflation of the compounds and formulations of the invention include compositions comprising solutions and/or suspensions in pharmaceutically acceptable, aqueous, or organic solvents, or mixture thereof and/or powders.

Examples of dosage forms suitable for buccal administration of the compounds and formulations of the invention include lozenges, tablets and the like, compositions comprising solutions and/or suspensions in pharmaceutically acceptable, aqueous, or organic solvents, or mixtures thereof and/or powders.

Examples of dosage forms suitable for sublingual administration of the compounds and formulations of the invention include lozenges, tablets and the like, compositions comprising solutions and/or suspensions in pharmaceutically acceptable, aqueous, or organic solvents, or mixtures thereof and/or powders.

Examples of dosage forms suitable for opthalmic administration of the compounds and formulations of the invention include inserts and/or compositions comprising solutions and/or suspensions in pharmaceutically acceptable, aqueous, or organic solvents.

Examples of controlled drug formulations useful for delivery of the compounds and formulations of the invention are found in, for example, Sweetman, S. C. (Ed.). Martindale. The Complete Drug Reference, 33rd Edition, Pharmaceutical Press, Chicago, 2002, 2483 pp.; Aulton, M. E. (Ed.) Pharmaceutics. The Science of Dosage Form Design. Churchill Livingstone, Edinburgh, 2000, 734 pp.; and, Ansel, H. C., Allen, L. V. and Popovich, N. G. Pharmaceutical Dosage Forms and Drug Delivery Systems, 7th Ed., Lippincott 1999, 676 pp. Excipients employed in the manufacture of drug delivery systems are described in various publications known to those skilled in the art including, for example, Kibbe, E. H. Handbook of Pharmaceutical Excipients, 3rd Ed., American Pharmaceutical Association, Washington, 2000, 665 pp. The USP also provides examples of modified-release oral dosage forms, including those formulated as tablets or capsules. See, for example, The United States Pharmacopeia 23/National Formulary 18, The United States Pharmacopeial Convention, Inc., Rockville Md., 1995 (hereinafter “the USP”), which also describes specific tests to determine the drug release capabilities of extended-release and delayed-release tablets and capsules. The USP test for drug release for extended-release and delayed-release articles is based on drug dissolution from the dosage unit against elapsed test time. Descriptions of various test apparatus and procedures may be found in the USP. The individual monographs contain specific criteria for compliance with the test and the apparatus and test procedures to be used. Examples have been given, for example for the release of aspirin from Aspirin Extended-release Tablets (for example, see: Ansel, H. C., Allen, L. V. and Popovich, N. G., Pharmaceutical Dosage Forms and Drug Delivery Systems, 7th Ed., Lippincott 1999, p. 237). Modified-release tablets and capsules must meet the USP standard for uniformity as described for conventional dosage units. Uniformity of dosage units may be demonstrated by either of two methods, weight variation or content uniformity, as described in the USP. Further guidance concerning the analysis of extended release dosage forms has been provided by the F.D.A. (See Guidance for Industry. Extended release oral dosage forms: development, evaluation, and application of in vitro/in vivo correlations. Rockville, Md.: Center for Drug Evaluation and Research, Food and Drug Administration, 1997).

Further examples of dosage forms of the invention include, but are not limited to modified-release (MR) dosage forms including delayed-release (DR) forms; prolonged-action (PA) forms; controlled-release (CR) forms; extended-release (ER) forms; timed-release (TR) forms; and long-acting (LA) forms. For the most part, these terms are used to describe orally administered dosage forms; however these terms may be applicable to any of the dosage forms, formulations, compositions and/or devices described herein. These formulations effect delayed total drug release for some time after drug administration, and/or drug release in small aliquots intermittently after administration, and/or drug release slowly at a controlled rate governed by the delivery system, and/or drug release at a constant rate that does not vary, and/or drug release for a significantly longer period than usual formulations.

Modified-release dosage forms of the invention include dosage forms having drug release features based on time, course, and/or location which are designed to accomplish therapeutic or convenience objectives not offered by conventional or immediate-release forms. See, for example, Bogner, R. H. Bioavailability and bioequivalence of extended-release oral dosage forms. U.S. Pharmacist 22 (Suppl.): 3-12 (1997); Scale-up of oral extended-release drug delivery systems: part I, an overview. Pharmaceutical Manufacturing 2: 23-27 (1985). Extended-release dosage forms of the invention include, for example, as defined by The United States Food and Drug Administration (FDA), a dosage form that allows a reduction in dosing frequency to that presented by a conventional dosage form, e.g., a solution or an immediate-release dosage form. See, for example, Bogner, R. H. Bioavailability and bioequivalence of extended-release oral dosage forms. US Pharmacist 22 (Suppl.): 3-12 (1997); Guidance for industry. Extended release oral dosage forms: development, evaluation, and application of the in vitro/in vivo correlations. Rockville, Md.: Center for Drug Evaluation and Research, Food and Drug Administration (1997). Repeat action dosage forms of the invention include, for example, forms that contain two single doses of medication, one for immediate release and the second for delayed release. Bi-layered tablets, for example, may be prepared with one layer of drug for immediate release with the second layer designed to release drug later as either a second dose or in an extended-release manner. Targeted-release dosage forms of the invention include, for example, formulations that facilitate drug release and which are directed towards isolating or concentrating a drug in a body region, tissue, or site for absorption or for drug action.

The invention in part provides dosage forms, formulations, devices and/or compositions and/or methods utilizing administration of dosage forms, formulations, devices and/or compositions incorporating one or more glyponectins, glyponectin agonists, adiponectins, and/or adiponectin agonists, including glycoisoforms and/or non-glycosylated isoforms, complexed with one or more suitable anions to yield complexes that are only slowly soluble in body fluids. One such example of modified release forms of one or more glyponectins, glyponectin agonists, adiponectins, and/or adiponectin agonists, including glycoisoforms and/or non-glycosylated isoforms is produced by the incorporation of the active agent or agents into certain complexes such as those formed with the anions of various forms of tannic acid (for example, see: Merck Index 12th Ed., 9221). Dissolution of such complexes may depend, for example, on the pH of the environment. This slow dissolution rate provides for the extended release of a glyponectin, glyponectin agonist, adiponectin, and/or adiponectin agonist, including glycoisoforms and/or non-glycosylated isoforms. For example, salts of tannic acid, and/or tannates, provide for this quality, and are expected to possess utility for the treatment of conditions in which glyponectin plays a role. Examples of equivalent products are provided by those having the tradename Rynatan (Wallace: see, for example, Madan, P. L., “Sustained release dosage forms,” U.S. Pharmacist 15: 39-50 (1990); Ryna-12 S, which contains a mixture of mepyramine tannate with phenylephrine tannate, Martindale 33rd Ed., 2080.4).

Also included in the invention are coated beads, granules or microspheres containing one or more glyponectins, glyponectin agonists, adiponectins, and/or adiponectin agonists, including glycoisoforms and/or non-glycosylated isoforms. Thus, the invention also provides a method to achieve modified release of one or more glyponectins, glyponectin agonists, adiponectins, and/or adiponectin agonists, including glycoisoforms and/or non-glycosylated isoforms, by incorporation of the drug into coated beads, granules, or microspheres. Such formulations of one or more glyponectins, glyponectin agonists, adiponectins, and/or adiponectin agonists, including glycoisoforms and/or non-glycosylated isoforms, have utility for the treatment of diseases in humans and other mammals in which glyponectin, glyponectin agonist, adiponectin, and/or adiponectin agonist, for example, glycoisoforms and/or non-glycosylated isoforms, is indicated. In such systems, the glyponectin, glyponectin agonist, adiponectin, and/or adiponectin agonist, including glycoisoforms and/or non-glycosylated isoforms, are distributed onto beads, pellets, granules or other particulate systems. Using conventional pan-coating or air-suspension coating techniques, a solution of the glyponectin, glyponectin agonist, adiponectin, and/or adiponectin agonist, substance is placed onto small inert nonpareil seeds or beads made of sugar and starch or onto microcrystalline cellulose spheres. The nonpareil seeds are most often in the 425 to 850 micrometer range whereas the microcrystalline cellulose spheres are available ranging from 170 to 600 micrometers (see Ansel, H. C., Allen, L. V. and Popovich, N. G., Pharmaceutical Dosage Forms and Drug Delivery Systems, 7th Ed., Lippincott 1999, p. 232). The microcrystalline spheres are considered more durable during production than sugar-based cores (see: Celphere microcrystalline cellulose spheres. Philadelphia: FMC Corporation, 1996). Methods for manufacture of microspheres suitable for drug delivery have been described (see, for example, Arshady, R. Microspheres and microcapsules: a survey of manufacturing techniques. 1: suspension and cross-linking. Polymer Eng Sci 30: 1746-1758 (1989); see also, Arshady, R., Micro-spheres and microcapsules: a survey of manufacturing techniques. 2: coacervation. Polymer Eng Sci 30: 905-914 (1990); see also: Arshady R., Microspheres and microcapsules: a survey of manufacturing techniques. 3: solvent evaporation. Polymer Eng Sci 30: 915-924 (1990). In instances in which the glyponectin, glyponectin agonist, adiponectin, and/or adiponectin agonist, including glycoisoforms and/or non-glycosylated isoforms dose is large, the starting granules of material may be composed of the glyponectin, glyponectin agonist, adiponectin, and/or adiponectin agonist, including glycoisoforms and/or non-glycosylated isoforms itself. Some of these granules may remain uncoated to provide immediate glyponectin, glyponectin agonist, adiponectin, and/or adiponectin agonist, including glycoisoforms and/or non-glycosylated isoforms release. Other granules (about two-thirds to three-quarters) receive varying coats of a lipid material such as beeswax, carnauba wax, glycerylmonostearate, cetyl alcohol, or a cellulose material such as ethylcellulose (infra). Subsequently, granules of different coating thickness are blended to achieve a mixture having the desired release characteristics. The coating material may be coloured with one or more dyes to distinguish granules or beads of different coating thickness (by depth of colour) and to provide distinctiveness to the product. When properly blended, the granules may be placed in capsules or tablets. Various coating systems are commercially available which are aqueous-based and which use ethylcellulose and plasticizer as the coating material (e.g., Aquacoat™ [FMC Corporation, Philadelphia] and Surerelease™ [Colorcon]; Aquacoat aqueous polymeric dispersion. Philadelphia: FMC Corporation, 1991; Surerelease aqueous controlled release coating system. West Point, Pa.: Colorcon, 1990; Butler, J., Cumming, I, Brown, J. et al., A novel multiunit controlled-release system, Pharm Tech 22: 122-138 (1998); Yazici, E., Oner, L., Kas, H. S. & Hincal, A. A., Phenytoin sodium microspheres: bench scale formulation, process characterization and release kinetics, Pharmaceut Dev Technol 1: 175-183 (1996)). Aqueous-based coating systems eliminate the hazards and environmental concerns associated with organic solvent-based systems. Aqueous and organic solvent-based coating methods have been compared (see, for example, Hogan, J. E. Aqueous versus organic solvent coating. Int J Pharm Tech Prod Manufacture 3: 17-20 (1982)). The variation in the thickness of the coats and in the type of coating materials used affects the rate at which the body fluids are capable of penetrating the coating to dissolve the glyponectin, glyponectin agonist, adiponectin, and/or adiponectin agonist, including glycoisoforms and/or non-glycosylated isoforms. Generally, the thicker the coat, the more resistant to penetration and the more delayed will be glyponectin, glyponectin agonist, adiponectin, and/or adiponectin agonist, including glycoisoforms and/or non- glycosylated isoforms release and dissolution. Typically, the coated beads are about 1 mm in diameter. They are usually combined to have three or four release groups among the more than 100 beads contained in the dosing unit (see Madan, P. L. Sustained release dosage forms. U.S. Pharmacist 15: 39-50 (1990)). This provides the different desired sustained or extended release rates and the targeting of the coated beads to the desired segments of the gastrointestinal tract. One example of this type of dosage form is the Spansule™ (SmithKline Beecham Corporation, U.K.). Examples of film-forming polymers which can be used in water-insoluble release-slowing intermediate layer(s) (to be applied to a pellet, spheroid or tablet core) include ethylcellulose, polyvinyl acetate, Eudragit® RS, Eudragit® RL, etc. (Each of Eudragit® RS and Eudragit® RL is an ammonio methacrylate copolymer). The release rate can be controlled not only by incorporating therein suitable water-soluble pore formers, such as lactose, mannitol, sorbitol, etc., but also by the thickness of the coating layer applied. Multi tablets may be formulated which include small spheroid-shaped compressed minitablets that may have a diameter of between 3 to 4 mm and can be placed in gelatin capsule shell to provide the desired pattern of glyponectin, glyponectin agonist, adiponectin, and/or adiponectin agonist, including glycoisoforms and/or non-glycosylated isoforms release. Each capsule may contain 8-10 minitablets, some uncoated for immediate release and others coated for extended release of the glyponectin, glyponectin agonist, adiponectin, and/or adiponectin agonist, including glycoisoforms and/or non-glycosylated isoforms.

A number of methods may be employed to generate modified-release dosage forms of one or more glyponectins, glyponectin agonists, adiponectins, and/or adiponectin agonists, including glycoisoforms and/or non-glycosylated isoforms suitable for oral administration to humans and other mammals. Two basic mechanisms are available to achieve modified release drug delivery. These are altered dissolution or diffusion of drugs and excipients. Within this context, for example, four processes may be employed, either simultaneously or consecutively. These are as follows: (i) hydration of the device (e.g., swelling of the matrix); (ii) diffusion of water into the device; (iii) controlled or delayed dissolution of the drug; and (iv) controlled or delayed diffusion of dissolved or solubilized drug out of the device. See, e.g., Examples 11, 12, 23, 24, 35, and 36 herein.

For orally administered dosage forms of the compounds and formulations of the invention, extended glyponectin, a glyponectin agonist, an adiponectin, and/or an adiponectin agonist, including glycoisoforms and/or non-glycosylated isoforms action, may be achieved by affecting the rate at which the glyponectin, glyponectin agonist, adiponectin, and/or adiponectin agonist, including glycoisoforms and/or non-glycosylated isoforms is released from the dosage form and/or by slowing the transit time of the dosage form through the gastrointestinal tract (see Bogner, R. H., Bioavailability and bioequivalence of extended-release oral dosage forms. US Pharmacist 22 (Suppl.): 3-12 (1997)). The rate of drug release from solid dosage forms may be modified by the technologies described below which, in general, are based on the following: 1) modifying drug dissolution by controlling access of biologic fluids to the drug through the use of barrier coatings; 2) controlling drug diffusion rates from dosage forms; and 3) chemically reacting or interacting between the drug substance or its pharmaceutical barrier and site-specific biological fluids. Systems by which these objectives are achieved are also provided herein. In one approach, employing digestion as the release mechanism, the glyponectin, glyponectin agonist, adiponectin, and/or adiponectin agonist, including glycoisoforms and/or non-glycosylated isoforms is either coated or entrapped in a substance that is slowly digested or dispersed into the intestinal tract. The rate of availability of the glyponectin, glyponectin agonist, adiponectin, and/or adiponectin agonist, including glycoisoforms and/or non-glycosylated isoforms is a function of the rate of digestion of the dispersible material. Therefore, the release rate, and thus the effectiveness of the glyponectin, glyponectin agonist, adiponectin, and/or adiponectin agonist, including glycoisoforms and/or non-glycosylated isoforms, varies from subject to subject depending upon the ability of the subject to digest the material.

A further form of slow release dosage form of the compounds and formulations of the invention is any suitable osmotic system where semipermeable membranes of for example cellulose acetate, cellulose acetate butyrate, cellulose acetate propionate, is used to control the release of glyponectin, glyponectin agonist, adiponectin, and/or adiponectin agonist, including glycoisoforms and/or non-glycosylated isoforms. These can be coated with aqueous dispersions of enteric lacquers without changing release rate. An example of such an osmotic system is an osmotic pump device, an example of which is the Oros™ device developed by Alza Inc. (U.S.A.). This system comprises a core tablet surrounded by a semi-permeable membrane coating having a 0.4 mm diameter hole produced by a laser beam. The core tablet has two layers, one containing the drug (the “active” layer) and the other containing a polymeric osmotic agent (the “push” layer). The core layer consists of active drug, filler, a viscosity modulator, and a solubilizer. The system operates on the principle of osmotic pressure. This system is suitable for delivery of a wide range of glyponectins, glyponectin agonists, adiponectins, and/or adiponectin agonists, including glycoisoforms and/or non-glycosylated isoforms. The coating technology is straightforward, and release is zero-order. When the tablet is swallowed, the semi-permeable membrane permits aqueous fluid to enter from the stomach into the core tablet, dissolving or suspending the glyponectin, glyponectin agonist, adiponectin, and/or adiponectin agonist. As pressure increases in the osmotic layer, it forces or pumps the glyponectin, glyponectin agonist, adiponectin, and/or adiponectin agonist, solution out of the delivery orifice on the side of the tablet. Only the glyponectin, glyponectin agonist, adiponectin, and/or adiponectin agonist solution (not the undissolved glyponectin, glyponectin agonist, adiponectin, and/or adiponectin agonist) is capable of passing through the hole in the tablet. The system is designed such that only a few drops of water are drawn into the tablet each hour. The rate of inflow of aqueous fluid and the function of the tablet depends on the existence of an osmotic gradient between the contents of the bi-layer and the fluid in the gastrointestinal tract. Delivery is essentially constant as long as the osmotic gradient remains unchanged. The glyponectin, glyponectin agonist, adiponectin, and/or adiponectin agonist release rate may be altered by changing the surface area, the thickness or composition of the membrane, and/or by changing the diameter of the glyponectin, glyponectin agonist, adiponectin, and/or adiponectin agonist release orifice. The glyponectin, glyponectin agonist, adiponectin, and/or adiponectin agonist release rate is not affected by gastrointestinal acidity, alkalinity, fed conditions, or gut motility. The biologically inert components of the tablet remain intact during gut transit and are eliminated in the feces as an insoluble shell. Other examples of the application of this technology are provided by Glucotrol XL Extended Release Tablets (Pfizer Inc.) and Procardia XL Extended Release Tablets (Pfizer Inc.; see, Martindale 33rd Ed., p. 2051.3).

The invention also provides devices for compounds and formulations of the invention that utilize monolithic matrices including, for example, slowly eroding or hydrophilic polymer matrices, in which one or more glyponectins, glyponectin agonists, adiponectins, and/or adiponectin agonists, including glycoisoforms and/or non-glycosylated isoforms is/are compressed or embedded.

Monolithic matrix devices comprising compounds and formulations of the invention include those formed using either of the following systems, for example: glyponectin, a glyponectin agonist, an adiponectin, and/or an adiponectin agonist dispersed in a soluble matrix, which become increasingly available as the matrix dissolves or swells; examples include hydrophilic colloid matrices, such as hydroxypropylcellulose (BP) or hydroxypropyl cellulose (USP); hydroxypropyl methylcellulose (HPMC; BP, USP); methylcellulose (MC; BP, USP); calcium carboxymethylcellulose (Calcium CMC; BP, USP); acrylic acid polymer or carboxy polymethylene (Carbopol) or Carbomer (BP, USP); or linear glycuronan polymers such as alginic acid (BP, USP), for example those formulated into microparticles from alginic acid (alginate)-gelatin hydrocolloid coacervate systems, or those in which liposomes have been encapsulated by coatings of alginic acid with poly-L-lysine membranes. Glyponectin, glyponectin agonist, adiponectin, and/or adiponectin agonist release occurs as the polymer swells, forming a matrix layer that controls the diffusion of aqueous fluid into the core and thus the rate of diffusion of glyponectin, glyponectin agonist, adiponectin, and/or adiponectin agonist from the system. In such systems, the rate of glyponectin, a glyponectin agonist, an adiponectin, and/or an adiponectin agonist release depends upon the tortuous nature of the channels within the gel, and the viscosity of the entrapped fluid, such that different release kinetics can be achieved, for example, zero-order, or first-order combined with pulsatile release. Where such gels are not cross-linked, there is a weaker, non-permanent association between the polymer chains, which relies on secondary bonding. With such devices, high loading of the glyponectin, glyponectin agonist, adiponectin, and/or adiponectin agonist, including glycoisoforms and/or non-glycosylated isoforms is achievable, and effective blending is frequent. Devices may contain 20-80% of glyponectin, a glyponectin agonist, an adiponectin, and/or an adiponectin agonist, including glycoisoforms and/or non-glycosylated isoforms (w/w), along with gel modifiers that can enhance glyponectin, glyponectin agonist, adiponectin, and/or adiponectin agonist diffusion; examples of such modifiers include sugars that can enhance the rate of hydration, ions that can influence the content of cross-links, and pH buffers that affect the level of polymer ionization. Hydrophilic matrix devices of the invention may also contain one or more of pH buffers, surfactants, counter-ions, lubricants such as magnesium stearate (BP, USP) and a glidant such as colloidal silicon dioxide (USP; colloidal anhydrous silica, BP) in addition to glyponectin, glyponectin agonist, adiponectin, and/or adiponectin agonist and hydrophilic matrix; (II) glyponectin, glyponectin agonist, adiponectin, and/or adiponectin agonist particles are dissolved in an insoluble matrix, from which the glyponectin, glyponectin agonist, adiponectin, and/or adiponectin agonist becomes available as solvent enters the matrix, often through channels, and dissolves the glyponectin, glyponectin agonist, adiponectin, and/or adiponectin agonist particles. Examples include systems formed with a lipid matrix, or insoluble polymer matrix, including preparations formed from Carnauba wax (BP; USP); medium-chain triglyceride such as fractionated coconut oil (BP) or triglycerida saturata media (PhEur); or cellulose ethyl ether or ethylcellulose (BP, USP). Lipid matrices are simple and easy to manufacture, and incorporate the following blend of powdered components: lipids (20-40% hydrophobic solids w/w) which remain intact during the release process; glyponectin, a glyponectin agonist, an adiponectin, and/or an adiponectin agonist, including glycoisoforms and non-glycosylated isoforms; channeling agent, such as sodium chloride or sugars, which leaches from the formulation, forming aqueous micro-channels (capillaries) through which solvent enters, and through which glyponectin, glyponectin agonist, adiponectin, and/or adiponectin agonist are released. In the alternative system, which employs an insoluble polymer matrix, the glyponectin, glyponectin agonist, adiponectin, and/or adiponectin agonist is embedded in an inert insoluble polymer and is released by leaching of aqueous fluid, which diffuses into the core of the device through capillaries formed between particles, and from which glyponectin, glyponectin agonist, adiponectin, and/or adiponectin agonist diffuses out of the device. The rate of release is controlled by the degree of compression, particle size, and the nature and relative content (w/w) of excipients. An example of such a device is that of Ferrous Gradumet (Martindale 33rd Ed., 1360.3). A further example of a suitable insoluble matrix is an inert plastic matrix. By this method, the glyponectin, glyponectin agonist, adiponectin, and/or adiponectin agonist is granulated with an inert plastic material such as polyethylene, polyvinyl acetate, or polymethacrylate, and the granulated mixture is then compressed into tablets. Once ingested, the glyponectin, glyponectin agonist, adiponectin, and/or adiponectin agonist is slowly released from the inert plastic matrix by diffusion (see, for example, Bodmeier, R. & Paeratakul, O., “Drug release from laminated polymeric films prepared from aqueous latexes,” J Pharm Sci 79:32-26 (1990); Laghoueg, N., et al., “Oral polymer-drug devices with a core and an erodible shell for constant drug delivery,” Int J Pharm 50: 133-139 (1989); Buckton, G., et al., “The influence of surfactants on drug release from acrylic matrices. Int J Pharm 74: 153-158 (1991)). The compression of the tablet creates the matrix or plastic form that retains its shape during the leaching of the glyponectin, glyponectin agonist, adiponectin, and/or adiponectin agonist and through its passage through the gastrointestinal tract. An immediate-release portion of the glyponectin, glyponectin agonist, adiponectin, and/or adiponectin agonist, may be compressed onto the surface of the tablet. The inert tablet matrix, expended of glyponectin, glyponectin agonist, adiponectin, and/or adiponectin agonist is excreted with the feces. An example of a successful dosage form of this type is Gradumet (Abbott; see, for example, Ferro-Gradumet, Martindale 33rd Ed., p. 1860.4).

Further examples of monolithic matrix devices of the invention have compositions and formulations of the invention incorporated in pendent attachments to a polymer matrix (see, for example, Scholsky, K. M. and Fitch, R. M., Controlled release of pendant bioactive materials from acrylic polymer colloids. J Controlled Release 3: 87-108 (1986)). In these devices, glyponectins, glyponectin agonists, adiponectins, and/or adiponectin agonists, including glycoisoforms and non-glycosylated isoforms, are attached by means of an ester linkage to poly(acrylate) ester latex particles prepared by aqueous emulsion polymerization.

Yet further examples of monolithic matrix devices of the invention incorporate dosage forms of the compositions and formulations of the invention in which the glyponectin, glyponectin agonist, adiponectin, and/or adiponectin agonist are bound to a biocompatible polymer by a labile chemical bond, e.g., polyanhydrides prepared from a substituted anhydride (itself prepared by reacting an acid chloride with the drug: methacryloyl chloride and the sodium salt of methoxy benzoic acid) have been used to form a matrix with a second polymer (Eudragit RL) which releases drug on hydrolysis in gastric fluid (see: Chafi, N., Montheard, J. P. & Vergnaud, J. M. Release of 2-aminothiazole from polymeric carriers. Int J Pharm 67: 265-274 (1992)).

In formulating a successful hydrophilic matrix system for the compositions and formulations of the invention, the polymer selected for use must form a gelatinous layer rapidly enough to protect the inner core of the tablet from disintegrating too rapidly after ingestion. As the proportion of polymer is increased in a formulation so is the viscosity of the gel formed with a resulting decrease in the rate of glyponectin, glyponectin agonist, adiponectin, and/or adiponectin agonist diffusion and release (see Formulating for controlled release with Methocel Premium cellulose ethers. Midland, Mich.: Dow Chemical Company, 1995). In general, 20% (w/w) of HPMC results in satisfactory rates of drug release for an extended-release tablet formulation; However, as with all formulations, consideration must be given to the possible effects of other formulation ingredients such as fillers, tablet binders, and disintegrants. An example of a proprietary product formulated using a hydrophilic matrix base of HPMC for extended drug release is that of Oramorph SR Tablets (Roxane; see Martindale 33rd Ed., p. 2014.4).

Two-layered tablets can be manufactured containing one or more of the compositions and formulations of the invention, with one layer containing the uncombined glyponectin, glyponectin agonist, adiponectin, and/or adiponectin agonist for immediate release and the other layer having the glyponectin, glyponectin agonist, adiponectin, and/or adiponectin agonist agonist imbedded in a hydrophilic matrix for extended-release. Three-layered tablets may also be similarly prepared, with both outer layers containing the glyponectin, glyponectin agonist, adiponectin, and/or adiponectin agonist for immediate release. Some commercial tablets are prepared with an inner core containing the extended-release portion of drug and an outer shell enclosing the core and containing drug for immediate release.

The invention also provides forming a complex between the compositions and formulations of the invention and an ion exchange resin, whereupon the complex may be tableted, encapsulated or suspended in an aqueous vehicle. Release of the glyponectin, glyponectin agonist, adiponectin, and/or adiponectin agonist is dependent on the local pH and electrolyte concentration such that the choice of ion exchange resin may be made so as to preferentially release the glyponectin, glyponectin agonist, adiponectin, and/or adiponectin agonist in a given region of the alimentary canal. Delivery devices incorporating such a complex are also provided. For example, a modified release dosage form of a glyponectin, glyponectin agonist, adiponectin, and/or adiponectin agonist, for example, one or more glycoisoforms and non-glycosylated isoforms can be produced by the incorporation of glyponectin, glyponectin agonist, adiponectin, and/or adiponectin agonist into complexes with an anion-exchange resin. Solutions of glyponectin, glyponectin agonist, adiponectin, and/or adiponectin agonist may be passed through columns containing an ion-exchange resin to form a complex by the replacement of H₃O⁺ ions. The resin-adiponectin or resin-glyponectin complex is then washed and may be tableted, encapsulated, or suspended in an aqueous vehicle. The release of the glyponectin, glyponectin agonist, adiponectin, and/or adiponectin agonist is dependent on the pH and the electrolyte concentration in the gastrointestinal fluid. Release is greater in the acidity of the stomach than in the less acidic environment of the small intestine. Alternative examples of this type of extended release preparation are provided by hydrocodone polistirex and chorpheniramine polistirex suspension (Medeva; Tussionex Pennkinetic Extended Release Suspension, see: Martindale 33rd Ed., p. 2145.2) and by phentermine resin capsules (Pharmanex; lonamin Capsules see: Martindale 33rd Ed., p. 1916.1). Such resin systems can additionally incorporate polymer barrier coating and bead technologies in addition to the ion-exchange mechanism. The initial dose comes from an uncoated portion, and the remainder from the coated beads, wherein release may be extended over a 12-hour period by ion exchange. The glyponectin, glyponectin agonist, adiponectin, and/or adiponectin agonist containing particles are minute, and may also be suspended to produce a liquid with extended-release characteristics, as well as solid dosage forms. Such preparations may also be suitable for administration, for example in depot preparations suitable for intramuscular injection.

The invention also provides a method to produce modified release preparations of one or more glyponectins, glyponectin agonists, adiponectins, and/or adiponectin agonists, for example, one or more glycoisoforms and/or non-glycosylated isoforms, by microencapsulation. Microencapsulation is a process by which solids, liquids, or even gasses may be encapsulated into microscopic size particles through the formation of thin coatings of “wall” material around the substance being encapsulated such as disclosed in U.S. Pat. Nos. 3,488,418; 3,391,416 and 3,155,590. Gelatin (BP, USP) is commonly employed as a wall-forming material in microencapsulated preparations, but synthetic polymers such as polyvinyl alcohol (USP), ethylcellulose (BP, USP), polyvinyl chloride, and other materials may also be used (see, for example, Zentner, G. M., Rork, G. S., and Himmelstein, K. J., Osmotic flow through controlled porosity films: an approach to delivery of water-soluble compounds, J Controlled Release 2: 217-229 (1985); Fites, A. L., Banker, G. S., and Smolen, V. F., Controlled drug release through polymeric films, J Pharm Sci 59: 610-613 (1970); Samuelov, Y., Donbrow, M., and Friedman, M., Sustained release of drugs from ethylcellulose-polyethylene glycol films and kinetics of drug release, J Pharm Sci 68: 325-329 (1979)).

Encapsulation begins with the dissolving of the prospective wall material, say gelatin, in water. One or more glyponectins, glyponectin agonists, adiponectins, and/or adiponectin agonists, for example, one or more glycoisoforms and/or non-glycosylated isoforms, is then added and the two-phase mixture is thoroughly stirred. With the material to be encapsulated broken up to the desired particle size, a solution of a second material is added. This additive material, for example, acacia, is chosen to have the ability to concentrate the gelatin (polymer) into tiny liquid droplets. These droplets (the coacervate) then form a film or coat around the particles of the solid glyponectin, glyponectin agonist, adiponectin, and/or adiponectin agonist as a consequence of the extremely low interfacial tension of the residual water or solvent in the wall material so that a continuous, tight, film-coating remains on the particle (see Ansel, H. C., Allen, L. V., and Popovich, N. G., Pharmaceutical Dosage Forms and Drug Delivery Systems, 7th Ed., Lippincott 1999, p. 233). The final dry microcapsules are free flowing, discrete particles of coated material. Of the total particle weight, the wall material usually represents between 2 and 20% (w/w). The coated particles are then admixed with tableting excipients and formed into dosage-sized tablets. Different rates of glyponectin, glyponectin agonist, adiponectin, and/or adiponectin agonist release may be obtained by changing the core-to-wall ratio, the polymer used for the coating, or the method of microencapsulation (for example, see: Yazici, E., Oner, L., Kas, H. S. & Hincal, A. A. Phenytoin sodium microspheres: bench scale formulation, process characterization and release kinetics. Pharmaceut Dev Technol 1996; 1: 175-183).

One of the advantages of microencapsulation is that the administered dose of one or more glyponectins, glyponectin agonists, adiponectins, and/or adiponectin agonists, for example, one or more glycoisoforms and/or non-glycosylated isoforms, is subdivided into small units that are spread over a large area of the gastrointestinal tract, which may enhance absorption by diminishing localized glyponectin, glyponectin agonist, adiponectin, and/or adiponectin agonist concentrations (see Yazici et al., supra). An example of a drug that is commercially available in a microencapsulated extended-release dosage form is potassium chloride (Micro-K Exten-caps, Wyeth-Ayerst, Martindale 33rd Ed., p 1968 1). Other useful approaches include those in which the glyponectin, glyponectin agonist, adiponectin, and/or adiponectin agonist is incorporated into polymeric colloidal particles or microencapsulates (microparticles, microspheres or nanoparticles) in the form or reservoir and matrix devices (see: Douglas, S. J., et al., “Nanoparticles in drug delivery,” C. R. C. Crit Rev Therap Drug Carrier Syst 3: 233-261 (1987); Oppenheim, R. C., “Solid colloidal drug delivery systems: nanoparticles,” Int J Pharm 8: 217-234 (1981); Higuchi, T., “Mechanism of sustained action medication: theoretical analysis of rate of release of solid drugs dispersed in solid matrices,” J Pharm Sci 52: 1145-1149 (1963)).

The invention also includes repeat action tablets containing one or more glyponectins, glyponectin agonists, adiponectins, and/or adiponectin agonists, for example, one or more glycoisoforms and/or non-glycosylated isoforms. These are prepared so that an initial dose of the glyponectin, glyponectin agonist, adiponectin, and/or adiponectin agonist is released immediately followed later by a second dose. The tablets may be prepared with the immediate-release dose in the tablet's outer shell or coating with the second dose in the tablet's inner core, separated by a slowly permeable barrier coating. In general, the glyponectin, glyponectin agonist, adiponectin, and/or adiponectin agonist from the inner core is exposed to body fluids and released 4 to 6 hours after administration. An example of this type of product is proved by Repetabs (Schering Inc.). Repeat action dosage forms are suitable for the administration of one or more glyponectins, glyponectin agonists, adiponectins, and/or adiponectin agonists for the indications noted herein.

The invention also includes delayed-release oral dosage forms containing one or more glyponectins, glyponectin agonists, adiponectins, and/or adiponectin agonists, for example, one or more glycoisoforms and/or non-glycosylated isoforms. The release of one or more glyponectins, glyponectin agonists, adiponectins, and/or adiponectin agonists, for example, one or more glycoisoforms and/or non-glycosylated isoforms, from an oral dosage form can be intentionally delayed until it reaches the intestine at least in part by way of, for example, enteric coating. Enteric coatings by themselves are not an efficient method for the delivery of glyponectin, a glyponectin agonist, an adiponectin, and/or an adiponectin agonist because of the inability of such coating systems to provide or achieve a sustained therapeutic effect after release onset. Enteric coats are designed to dissolve or break down in an alkaline environment. The presence of food may increase the pH of the stomach. Therefore, the concurrent administration of enteric-coated glyponectin, glyponectin agonist, adiponectin, and/or adiponectin agonist with food or the presence of food in the stomach may lead to dose dumping and unwanted secondary effects. Furthermore, in the event of gastrointestinal side effects, it would be desirable to have a glyponectin, glyponectin agonist, adiponectin, and/or adiponectin agonist form that is capable of providing the controlled delivery of a glyponectin, a glyponectin agonist, an adiponectin, and/or an adiponectin agonist in a predictable manner over a long period of time. See, e.g., Examples, 11, 12, 23, 24, 35, and 36 herein.

Enteric coatings have application in the present invention when combined or incorporated with one or more of the other dose delivery formulations or devices described herein. This form of delivery conveys the advantage of minimizing the gastric irritation that may be caused in some subjects by glyponectin, glyponectin agonist, adiponectin, and/or adiponectin agonist such as, for example, a glycoisoform and/or a non-glycosylated isoform. The enteric coating may be time-dependent, pH-dependent where it breaks down in the less acidic environment of the intestine and erodes by moisture over time during gastrointestinal transit, or enzyme-dependent where it deteriorates due to the hydrolysis-catalyzing action of intestinal enzymes (see, for example, Muhammad, N. A., et al., “Modifying the release properties of Eudragit L30D,” Drug Dev Ind Pharm., 17: 2497-2509 (1991)). Among the many agents used to enteric coat tablets and capsules known to those skilled in the art are fats including triglycerides, fatty acids, waxes, shellac, and cellulose acetate phthalate although further examples of enteric coated preparations can be found in the USP. See, e.g., Examples 12, 24, and 36 herein.

The invention also provides devices incorporating one or more glyponectins, glyponectin agonists, adiponectins, and/or adiponectin agonists, for example, one or more glycoisoforms and/or non-glycosylated isoforms, in a membrane-control system. Such devices comprise a rate-controlling membrane enclosing a glyponectin, glyponectin agonist, adiponectin, and/or adiponectin agonist reservoir. Following oral administration the membrane gradually becomes permeable to aqueous fluids, but does not erode or swell. The glyponectin, glyponectin agonist, adiponectin, and/or adiponectin agonist reservoir may be composed of a conventional tablet, or a microparticle pellet containing multiple units that do not swell following contact with aqueous fluids. The cores dissolve without modifying their internal osmotic pressure, thereby avoiding the risk of membrane rupture, and typically comprise 60:40 mixtures of lactulose: microcrystalline cellulose (w/w). Active drug(s) is/are released through a two-phase process, comprising diffusion of aqueous fluids into the matrix, followed by diffusion of the glyponectin, glyponectin agonist, adiponectin, and/or adiponectin agonist out of the matrix. Multiple-unit membrane-controlled systems typically comprise more than one discrete unit. They can contain discrete spherical beads individually coated with rate-controlling membrane and may be encapsulated in a hard gelatin shell (examples of such preparations include Contac 400; Martindale 33rd Ed., 1790.1 and Feospan; Martindale 33rd Ed., p. 1859.4). Alternatively, multiple-unit membrane-controlled systems may be compressed into a tablet (for example, Suscard; Martindale 33rd Ed., p. 2115.1). Alternative implementations of this technology include devices in which the glyponectin, glyponectin agonist, adiponectin, and/or adiponectin agonist is coated around inert sugar spheres, and devices prepared by extrusion spheronization employing a conventional matrix system. Advantages of such systems include the more consistent gastro-intestinal transit rate achieved by multiple-unit systems, and the fact that such systems infrequently suffer from catastrophic dose dumping. They are also ideal for the delivery of more than one drug at a time, as disclosed herein.

An example of a sustained release dosage form of one or more compounds and formulations of the invention is a matrix formation, such a matrix formation taking the form of film coated spheroids containing as active ingredient one or more glyponectins, glyponectin agonists, adiponectins, and/or adiponectin agonists, for example, one or more glycoisoforms and/or non-glycosylated isoforms and a non water-soluble spheronising agent. The term “spheroid” is known in the pharmaceutical art and means spherical granules having a diameter usually of between 0.01 mm and 4 mm. The spheronising agent may be any pharmaceutically acceptable material that, together with the glyponectin, glyponectin agonist, adiponectin, and/or adiponectin agonist, can be spheronised to form spheroids. Microcrystalline cellulose is preferred. Suitable microcrystalline cellulose includes, for example, the material sold as Avicel PH 101 (Trade Mark, FMC Corporation). The film-coated spheroids may contain between 70% and 99% (by wt), especially between 80% and 95% (by wt), of the spheronising agent, especially microcrystalline cellulose. In addition to the active ingredient and spheronising agent, the spheroids may also contain a binder. Suitable binders, such as low viscosity, water soluble polymers, will be well known to those skilled in the pharmaceutical art. A suitable binder is, in particular polyvinylpyrrolidone in various degrees of polymerization. However, water-soluble hydroxy lower alkyl celluloses, such as hydroxy propyl cellulose, are preferred. Additionally (or alternatively) the spheroids may contain a water insoluble polymer, especially an acrylic polymer, an acrylic copolymer, such as a methacrylic acid-ethyl acrylate copolymer, or ethyl cellulose. Other thickening agents or binders include: the lipid type, among which are vegetable oils (cotton seed, sesame and groundnut oils) and derivatives of these oils (hydrogenated oils such as hydrogenated castor oil, glycerol behenate, the waxy type such as natural carnauba wax or natural beeswax, synthetic waxes such as cetyl ester waxes, the amphiphilic type such as polymers of ethylene oxide (polyoxyethylene glycol of high molecular weight between 4000 and 100000) or propylene and ethylene oxide copolymers (poloxamers), the cellulosic type (semisynthetic derivatives of cellulose, hydroxypropylmethylcellulose, hydroxypropylcellulose, hydroxymethylcellulose, of high molecular weight and high viscosity, gum) or any other polysaccharide such as alginic acid, the polymeric type such as acrylic acid polymers (such as carbomers), and the mineral type such as colloidal silica and bentonite.

Suitable diluents for the glyponectins, glyponectin agonists, adiponectins, and/or adiponectin agonists in the pellets, spheroids or core are, e.g., microcrystalline cellulose, lactose, dicalcium phosphate, calcium carbonate, calcium sulphate, sucrose, dextrates, dextrin, dextrose, dicalcium phosphate dihydrate, kaolin, magnesium carbonate, magnesium oxide, maltodextrin, cellulose, microcrystalline cellulose, sorbitol, starches, pregelatinized starch, talc, tricalcium phosphate and lactose. Suitable lubricants are e.g., magnesium stearate and sodium stearyl fumarate. Suitable binding agents include, e.g., hydroxypropyl methylcellulose, polyvidone, and methylcellulose.

Suitable binders that may be included are: gum arabic, gum tragacanth, guar gum, alginic acid, sodium alginate, sodium carboxymethylcellulose, dextrin, gelatin, hydroxyethylcellulose, hydroxypropylcellulose, liquid glucose, magnesium and aluminum. Suitable disintegrating agents are starch, sodium starch glycolate, crospovidone and croscarmalose sodium. Suitable surface active are Poloxamer 188®, polysorbate 80 and sodium lauryl sulfate. Suitable flow aids are talc colloidal anhydrous silica. Suitable lubricants that may be used are glidants (such as anhydrous silicate, magnesium trisilicate, magnesium silicate, cellulose, starch, talc or tricalcium phosphate) or alternatively antifriction agents (such as calcium stearate, hydrogenated vegetable oils, paraffin, magnesium stearate, polyethylene glycol, sodium benzoate, sodium lauryl sulphate, fumaric acid, stearic acid or zinc stearate and talc). Suitable water-soluble polymers are PEG with molecular weights in the range 1000 to 6000.

Delayed release of the composition or formulation of the invention may be achieved through the use of a tablet, pellet, spheroid or core itself, which besides having a filler and binder, other ancillary substances, in particular lubricants and nonstick agents, and disintegrants. Examples of lubricants and nonstick agents are higher fatty acids and their alkali metal and alkaline-earth-metal salts, such as calcium stearate. Suitable disintegrants are, in particular, chemically inert agents, for example, cross-linked polyvinylpyrrolidone, cross-linked sodium carboxymethylcelluloses, and sodium starch glycolate.

Yet further embodiments of the invention include formulations of one or more glyponectins, glyponectin agonists, adiponectins, and/or adiponectin agonists, for example, one or more glycoisoforms and/or non-glycosylated isoforms, incorporated into transdermal drug delivery systems, such as those described in: Transdermal Drug Delivery Systems, Chapter 10. In: Ansel, H. C., Allen, L. V. and Popovich, N. G. Pharmaceutical Dosage Forms and Drug Delivery Systems, 7th Ed., Lippincott 1999, pp. 263-278). Transdermal drug delivery systems facilitate the passage of therapeutic quantities of drug substances through the skin and into the systemic circulation to exert systemic effects, as originally described (see Stoughton, R. D. Percutaneous absorption, Toxicol Appl Pharmacol 7: 1-8 (1965)). Evidence of percutaneous drug absorption may be found through measurable blood levels of the drug, detectable excretion of the drug and/or its metabolites in the urine, and through the clinical response of the subject to its administration. For transdermal drug delivery, it is considered ideal if the drug penetrates through the skin to the underlying blood supply without drug build up in the dermal layers (Black, C. D., “Transdermal drug delivery systems,” U.S. Pharm 1: 49 (1982)). Formulations of drugs suitable for trans-dermal delivery are known to those skilled in the art, and are described in references such as Ansel et al., (supra). Methods known to enhance the delivery of drugs by the percutaneous route include chemical skin penetration enhancers, which increase skin permeability by reversibly damaging or otherwise altering the physicochemical nature of the stratum corneum to decrease its resistance to drug diffusion (see Shah, V., Peck, C. C., and Williams, R. L., Skin penetration enhancement: clinical pharmacological and regulatory considerations, In: Walters, K. A. and Hadgraft, J. (Eds.) Pharmaceutical skin penetration enhancement. New York: Dekker, 1993). Among effective alterations are increased hydration of the stratum corneum and/or a change in the structure of the lipids and lipoproteins in the intercellular channels brought about through solvent action or denaturation (see Walters K. A., “Percutaneous absorption and transdermal therapy,” Pharm Tech 10: 30-42 (1986)). Skin penetration enhancers suitable for formulation with glyponectins, glyponectin agonists, adiponectins, and/or adiponectin agonists in transdermal drug delivery systems may be chosen from the following list: acetone, laurocapram, dimethylacetamide, dimethylformamide, dimethylsulphoxide, ethanol, oleic acid, polyethylene glycol, propylene glycol and sodium lauryl sulphate. Further skin penetration enhancers may be found in publications known to those skilled in the art (see, for example, Osborne, D. W., & Henke, J. J., “Skin penetration enhancers cited in the technical literature,” Pharm Tech 21: 50-66 (1997); Rolf, D., “Chemical and physical methods of enhancing transdermal drug delivery,” Pharm Tech 12: 130-139 (1988)).

In addition to chemical means, there are physical methods that enhance transdermal drug delivery and penetration of the compounds and formulations of the invention. These include iontophoresis and sonophoresis. Iontophoresis involves the delivery of charged chemical compounds across the skin membrane using an applied electrical field. Such methods have proven suitable for delivery of a number of drugs. Accordingly, another embodiment of the invention comprises one or more glyponectins, glyponectin agonists, adiponectins, and/or adiponectin agonists, for example, one or more glycoisoforms and/or non-glycosylated isoforms, formulated in such a manner suitable for administration by iontophoresis or sonophoresis. Formulations suitable for administration by iontophoresis or sonophoresis may be in the form of gels, creams, or lotions. Transdermal delivery, methods or formulations of the invention, may utilize, among others, monolithic delivery systems, drug-impregnated adhesive delivery systems (e.g., the Latitude™ drug-in-adhesive system from 3M), active transport devices and membrane-controlled systems. Monolithic systems of the invention incorporate a glyponectin, glyponectin agonist, adiponectin, and/or adiponectin agonist matrix, comprising a polymeric material in which the glyponectin, glyponectin agonist, adiponectin, and/or adiponectin agonist is dispersed between backing and frontal layers. Drug impregnated adhesive delivery systems comprise an adhesive polymer in which one or more compositions and formulations of the invention and any excipients are incorporated into the adhesive polymer. Active transport devices incorporate a glyponectin, glyponectin agonist, adiponectin, and/or adiponectin agonist reservoir, often in liquid or gel form, a membrane that may be rate controlling, and a driving force to propel the glyponectin, glyponectin agonist, adiponectin, and/or adiponectin agonist across the membrane. Membrane-controlled transdermal systems of the invention comprise a glyponectin, glyponectin agonist, adiponectin, and/or adiponectin agonist reservoir(s), often in liquid or gel form, a membrane that may be rate controlling and backing, adhesive and/or protecting layers. Transdermal delivery dosage forms of the invention include those which substitute the glyponectin, glyponectin agonist, adiponectin, and/or adiponectin agonist, for the diclofenic or other pharmaceutically acceptable salt thereof referred to in the transdermal delivery systems disclosed in, by way of example, U.S. Pat. Nos. 6,193,996, and 6,262,121.

Formulations and/or compositions for topical administration of one or more compositions and formulations of the invention ingredient can be prepared as an admixture or other pharmaceutical formulation to be applied in a wide variety of ways including, but are not limited to, lotions, creams gels, sticks, sprays, ointments and pastes. These product types may comprise several types of formulations including, but not limited to solutions, emulsions, gels, solids, and liposomes. If the topical composition of the invention is formulated as an aerosol and applied to the skin as a spray-on, a propellant may be added to a solution composition. Suitable propellants as used in the art can be utilized. By way of example of topical administration of an active agent, reference is made to U.S. Pat. Nos. 5,602,125, 6,426,362 and 6,420,411.

Also included in the dosage forms in accordance with the present invention are any variants of the oral dosage forms that are adapted for suppository or other parenteral use. When rectally administered in the form of suppositories, for example, these compositions may be prepared by mixing one or more compounds and formulations of the invention with a suitable non-irritating excipient, such as cocoa butter, synthetic glyceride esters or polyethylene glycols, which are solid at ordinary temperatures, but liquify and/or dissolve in the rectal cavity to release the glyponectin, glyponectin agonist, adiponectin, and/or adiponectin agonist. Suppositories are generally solid dosage forms intended for insertion into body orifices including rectal, vaginal and occasionally urethrally and can be long acting or slow release. Suppositories include a base that can include, but is not limited to, materials such as alginic acid, which will prolong the release of the pharmaceutically acceptable active ingredient over several hours (5-7). Such bases can be characterized into two main categories and a third miscellaneous group: 1) fatty or oleaginous bases, 2) water-soluble or water-miscible bases and 3) miscellaneous bases, generally combinations of lipophilic and hydrophilic substances. Fatty or oleaginous bases include hydrogenated fatty acids of vegetable oils such as palm kernel oil and cottonseed oil, fat-based compound containing compounds of glycerin with the higher molecular weight fatty acids such as palmitic and stearic acids, cocoa butter is also used where phenol and chloral hydrate lower the melting point of cocoa butter when incorporated, solidifying agents like cetyl esters wax (about 20%) or beeswax (about 4%) may be added to maintain a solid suppository. Other bases include other commercial products such as Fattibase (triglycerides from palm, palm kernel and coconut oils with self-emulsifying glycerol monostearate and poloxyl stearate), Wecobee and Witepsol bases. Water-soluble bases are generally glycerinated gelatin and water-miscible bases are generally polyethylene glycols. The miscellaneous bases include mixtures of the oleaginous and water-soluble or water-miscible materials. An example of such a base in this group is polyoxyl 40 stearate and polyoxyethylene diols and the free glycols.

Transmucosal administration of the compounds and formulations of the invention may utilize any mucosal membrane but commonly utilizes the nasal, buccal, vaginal and rectal tissues.

Formulations suitable for nasal administration of the compounds and formulations of the invention may be administered in a liquid form, for example, nasal spray, nasal drops, or by aerosol administration by nebulizer, including aqueous or oily solutions of the glyponectin, glyponectin agonist, adiponectin, and/or adiponectin agonist. Formulations for nasal administration, wherein the carrier is a solid, include a coarse powder having a particle size, for example, of less than about 100 microns, preferably less, most preferably one or two times per day than about 50 microns, which is administered in the manner in which snuff is taken, i.e., by rapid inhalation through the nasal passage from a container of the powder held close up to the nose. Compositions in solution may be nebulized by the use of inert gases and such nebulized solutions may be breathed directly from the nebulizing device or the nebulizing device may be attached to a facemask, tent or intermittent positive-pressure breathing machine. Solutions, suspensions or powder compositions of the glyponectin, glyponectin agonist, adiponectin, and/or adiponectin agonist may be administered orally or nasally from devices that deliver the formulation in an appropriate manner. Formulations of the invention may be prepared as aqueous solutions for example in saline, solutions employing benzyl alcohol or other suitable preservatives, absorption promoters to enhance bio-availability, fluorocarbons, and/or other solubilising or dispersing agents known in the art.

The invention provides extended-release formulations containing one or more glyponectins, glyponectin agonists, adiponectins, and/or adiponectin agonists, for example, one or more glycoisoforms and/or non-glycosylated isoforms, for parenteral administration. Extended rates of glyponectin, glyponectin agonist, adiponectin, and/or adiponectin agonist action following injection may be achieved in a number of ways, including the following: crystal or amorphous glyponectin, glyponectin agonist, adiponectin, and/or adiponectin agonist forms having prolonged dissolution characteristics; slowly dissolving chemical complexes of the glyponectin, glyponectin agonist, adiponectin, and/or adiponectin agonist formulation; solutions or suspensions of glyponectins, glyponectin agonists, adiponectins, and/or adiponectin agonists in slowly absorbed carriers or vehicles (as oleaginous); increased particle size of glyponectins, glyponectin agonists, adiponectins, and/or adiponectin agonists in suspension; or, by injection of slowly eroding microspheres of glyponectin, glyponectin agonist, adiponectin, and/or adiponectin agonist (for example, see: Friess, W., Lee, G. and Groves, M. J. Insoluble collagen matrices for prolonged delivery of proteins. Pharmaceut Dev Technol 1: 185-193 (1996)). The duration of action of the various forms of insulin, for example, is based in part on its physical form (amorphous or crystalline), complex formation with added agents, and its dosage form (solution of suspension).

The compositions of the invention can be formulated into a pharmaceutical composition suitable for administration to a patient.

The composition can be prepared according to conventional methods by dissolving or suspending an amount of the glyponectin, glyponectin agonist, adiponectin, and/or adiponectin agonist ingredient in a diluent.

The glyponectin, glyponectin agonist, adiponectin, and/or adiponectin agonist can be provided and administered in forms suitable for once-a-day dosing. An acetate, phosphate, citrate or glutamate buffer may be added allowing a pH of the final composition to be from about 5.0 to about 9.5; optionally a carbohydrate or polyhydric alcohol tonicifier and, a preservative selected from the group consisting of m-cresol, benzyl alcohol, methyl, ethyl, propyl and butyl parabens and phenol may also be added. Water for injection, tonicifying agents such as sodium chloride, as well as other excipients, may also be present, if desired. For parenteral administration, formulations are isotonic or substantially isotonic to avoid irritation and pain at the site of administration.

The terms buffer, buffer solution and buffered solution, when used with reference to hydrogen-ion concentration or pH, refer to the ability of a system, particularly an aqueous solution, to resist a change of pH on adding acid or alkali, or on dilution with a solvent. Characteristic of buffered solutions, which undergo small changes of pH on addition of acid or base, is the presence either of a weak acid and a salt of the weak acid, or a weak base and a salt of the weak base. An example of the former system is acetic acid and sodium acetate. The change of pH is slight as long as the amount of hydroxyl ion added does not exceed the capacity of the buffer system to neutralize it.

Maintaining the pH of the formulation in the range of approximately 5.0 to 9.5 can enhance the stability of the parenteral formulation of the present invention. Other pH ranges, for example, include, 5.5 to 9.0, or 6.0 to 8.5, or 6.5 to 8.0, or 7.0 to 7.5.

The buffer used in the practice of the present invention is selected from any of the following, for example, an acetate buffer, a phosphate buffer or glutamate buffer, the most preferred buffer being a phosphate buffer.

Carriers or excipients can also be used to facilitate administration of the compositions and formulations of the invention. Examples of carriers and excipients include calcium carbonate, calcium phosphate, various sugars such as lactose, glucose, or sucrose, or types of starch, cellulose derivatives, gelatin, polyethylene glycols and physiologically compatible solvents.

A stabilizer may be included in the formulations of the invention, but will generally not be needed. If included, however, a stabilizer useful in the practice of the invention is a carbohydrate or a polyhydric alcohol. The polyhydric alcohols include such compounds as sorbitol, mannitol, glycerol, xylitol, and polypropylene/ethylene glycol copolymer, as well as various polyethylene glycols (PEG) of molecular weight 200, 400, 1450, 3350, 4000, 6000, and 8000). The carbohydrates include, for example, mannose, ribose, trehalose, maltose, inositol, lactose, galactose, arabinose, or lactose.

The United States Pharmacopeia (USP) states that anti-microbial agents in bacteriostatic or fungistatic concentrations must be added to preparations contained in multiple dose containers. They must be present in adequate concentration at the time of use to prevent the multiplication of microorganisms inadvertently introduced into the preparation while withdrawing a portion of the contents with a hypodermic needle and syringe, or using other invasive means for delivery, such as pen injectors. Antimicrobial agents should be evaluated to ensure compatibility with all other components of the formula, and their activity should be evaluated in the total formula to ensure that a particular agent that is effective in one formulation is not ineffective in another. It is not uncommon to find that a particular agent will be effective in one formulation but not effective in another formulation.

A preservative is, in the common pharmaceutical sense, a substance that prevents or inhibits microbial growth and may be added to a pharmaceutical formulation for this purpose to avoid consequent spoilage of the formulation by microorganisms. While the amount of the preservative is not great, it may nevertheless affect the overall stability of the glyponectin, glyponectin agonist, adiponectin, and/or adiponectin agonist.

While the preservative for use in the practice of the invention can range from 0.005 to 1.0% (w/v), the preferred range for each preservative, alone or in combination with others, is: benzyl alcohol (0.1-1.0%), or m-cresol (0.1-0.6%), or phenol (0.1-0.8%) or combination of methyl (0.05-0.25%) and ethyl or propyl or butyl (0.005%-0.03%) parabens. The parabens are lower alkyl esters of para-hydroxybenzoic acid.

A detailed description of each preservative is set forth in “Remington's Pharmaceutical Sciences” as well as Pharmaceutical Dosage Forms: Parenteral Medications, Vol. 1, 1992, Avis et al. For these purposes, the glyponectin, glyponectin agonist, adiponectin, and/or adiponectin agonist may be administered parenterally (including subcutaneous injections, intravenous, intramuscular, intradermal injection or infusion techniques) or by inhalation spray in dosage unit formulations containing conventional non-toxic pharmaceutically acceptable carriers, adjuvants and vehicles.

If desired, the parenteral formulation may be thickened with a thickening agent such as a methylcellulose. The formulation may be prepared in an emulsified form, either water in oil or oil in water. Any of a wide variety of pharmaceutically acceptable emulsifying agents may be employed including, for example, acacia powder, a non-ionic surfactant or an ionic surfactant.

It may also be desirable to add suitable dispersing or suspending agents to the pharmaceutical formulation. These may include, for example, aqueous suspensions such as synthetic and natural gums, e.g., tragacanth, acacia, alginate, dextran, sodium carboxymethylcellulose, methylcellulose, polyvinyl-pyrrolidone or gelatin.

It is possible that other ingredients may be present in the parenteral pharmaceutical formulation of the invention. Such additional ingredients may include wetting agents, oils (e.g., a vegetable oil such as sesame, peanut or olive), analgesic agents, emulsifiers, antioxidants, bulking agents, tonicity modifiers, metal ions, oleaginous vehicles, proteins (e.g., human serum albumin, gelatin or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Containers and kits are also a part of a composition and may be considered a component. Therefore, the selection of a container is based on a consideration of the composition of the container, as well as of the ingredients, and the treatment to which it will be subjected.

Regarding pharmaceutical formulations, see also, Pharmaceutical Dosage Forms: Parenteral Medications, Vol. 1, 2nd ed., Avis et al., Eds., Mercel Dekker, New York, N.Y. 1992.

Suitable routes of parenteral administration include intramuscular, intravenous, subcutaneous, intraperitoneal, subdermal, intradermal, intraarticular, intrathecal and the like. Mucosal delivery is also permissible. The dose and dosage regimen will depend upon the weight and health of the subject.

In addition to the above means of achieving extended drug action, the rate and duration of glyponectin, glyponectin agonist, adiponectin, and/or adiponectin agonist delivery may be controlled by, for example by using mechanically controlled drug infusion pumps.

The glyponectins, glyponectin agonists, adiponectins, and/or adiponectin agonists, such as, for example, glycoisoforms and/or non-glycosylated isoforms, can be administered in the form of a depot injection that may be formulated in such a manner as to permit a sustained release of the glyponectin, glyponectin agonist, adiponectin, and/or adiponectin agonist. The glyponectin, glyponectin agonist, adiponectin, and/or adiponectin agonist can be compressed into pellets or small cylinders and implanted subcutaneously or intramuscularly. The pellets or cylinders may additionally be coated with a suitable biodegradable polymer chosen so as to provide a desired release profile. The glyponectin, glyponectin agonist, adiponectin, and/or adiponectin agonist may alternatively be micropelleted. The glyponectin, glyponectin agonist, adiponectin, and/or adiponectin agonist micropellets using bioacceptable polymers can be designed to allow release rates to be manipulated to provide a desired release profile. Alternatively, injectable depot forms can be made by forming microencapsulated matrices of the glyponectin, glyponectin agonist, adiponectin, and/or adiponectin agonist in biodegradable polymers such as polylactide-polyglycolide. Depending on the ratio of glyponectin, glyponectin agonist, adiponectin, and/or adiponectin agonist to polymer, and the nature of the particular polymer employed, the rate of glyponectin, glyponectin agonist, adiponectin, and/or adiponectin agonist release can be controlled. Examples of other biodegradable polymers include poly(orthoesters) and poly(anhydrides). Depot injectable formulations can also be prepared by entrapping the glyponectin, glyponectin agonist, adiponectin, and/or adiponectin agonist in liposomes, examples of which include unilamellar vesicles, large unilamellar vesicles and multilamellar vesicles. Liposomes can be formed from a variety of phospholipids, such as cholesterol, stearyl amine or phosphatidylcholines. Depot injectable formulations can also be prepared by entrapping the glyponectin, glyponectin agonist, adiponectin, and/or adiponectin agonist in microemulsions that are compatible with body tissue. By way of example reference is made to U.S. Pat. Nos. 6,410,041 and 6,362,190.

The invention in part provides infusion dose delivery formulations and devices, including but not limited to implantable infusion devices for delivery of compositions and formulations of the invention. Implantable infusion devices may employ inert material such as biodegradable polymers listed above or synthetic silicones, for example, cylastic, silicone rubber or other polymers manufactured by the Dow-Coming Corporation. The polymer may be loaded with glyponectin, glyponectin agonist, adiponectin, and/or adiponectin agonist and any excipients. Implantable infusion devices may also comprise a coating of, or a portion of, a medical device wherein the coating comprises the polymer loaded with glyponectin, glyponectin agonist, adiponectin, and/or adiponectin agonist and any excipient. Such an implantable infusion device may be prepared as disclosed in U.S. Pat. No. 6,309,380 by coating the device with an in vivo biocompatible and biodegradable or bioabsorbable or bioerodible liquid or gel solution containing a polymer with the solution comprising a desired dosage amount of glyponectin, glyponectin agonist, adiponectin, and/or adiponectin agonist and any excipients. The solution is converted to a film adhering to the medical device thereby forming the implantable glyponectin-, glyponectin agonist-, adiponectin-, and/or adiponectin agonist-deliverable medical device.

An implantable infusion device may also be prepared by the in situ formation of a glyponectin, glyponectin agonist, adiponectin, and/or adiponectin agonist containing solid matrix as disclosed in U.S. Pat. No. 6,120,789, herein incorporated in its entirety. Implantable infusion devices may be passive or active. An active implantable infusion device may comprise a glyponectin, glyponectin agonist, adiponectin, and/or adiponectin agonist reservoir, a means of allowing the glyponectin, glyponectin agonist, adiponectin, and/or adiponectin agonist to exit the reservoir, for example a permeable membrane, and a driving force to propel the glyponectin, glyponectin agonist, adiponectin, and/or adiponectin agonist from the reservoir. Such an active implantable infusion device may additionally be activated by an extrinsic signal, such as that disclosed in WO 02/45779, wherein the implantable infusion device comprises a system configured to deliver the glyponectin, glyponectin agonist, adiponectin, and/or adiponectin agonist comprising an external activation unit operable by a user to request activation of the implantable infusion device, including a controller to reject such a request prior to the expiration of a lockout interval. Examples of an active implantable infusion device include implantable drug pumps. Implantable drug pumps include, for example, miniature, computerized, programmable, refillable drug delivery systems with an attached catheter that inserts into a target organ system, usually the spinal cord or a vessel. See Medtronic Inc. Publications: UC9603124EN NP-2687, 1997; UC199503941b EN NP-2347 182577-101, 2000; UC199801017a EN NP3273a 182600-101, 2000; UC200002512 EN NP4050, 2000; UC199900546bEN NP-3678EN, 2000. Minneapolis, Minn.: Medtronic Inc; 1997-2000. Many pumps have 2 ports: one into which drugs can be injected and the other that is connected directly to the catheter for bolus administration or analysis of fluid from the catheter. Implantable drug infusion pumps (SynchroMed EL and Synchromed programmable pumps; Medtronic) are indicated for long-term intrathecal infusion of morphine sulfate for the treatment of chronic intractable pain; intravascular infusion of floxuridine for treatment of primary or metastatic cancer; intrathecal injection (baclofen injection) for severe spasticity; long-term epidural infusion of morphine sulfate for treatment of chronic intractable pain; long-term intravascular infusion of doxorubicin, cisplatin, or methotrexate for the treatment or metastatic cancer; and long-term intravenous infusion of clindamycin for the treatment of osteomyelitis. Such pumps may also be used for the long-term infusion of one or more glyponectins, glyponectin agonists,. adiponectins, and/or adiponectin agonists, for example, one or more glycoisoforms and/or non-glycosylated isoforms, at a desired amount for a desired number of doses or steady state administration. One form of a typical implantable drug infusion pump (Synchromed EL programmable pump; Medtronic) is titanium covered and roughly disk shaped, measures 85.2 mm in diameter and 22.86 mm in thickness, weighs 185 g, has a drug reservoir of 10 mL, and runs on a lithium thionyl-chloride battery with a 6- to 7-year life, depending on use. The downloadable memory contains programmed drug delivery parameters and calculated amount of drug remaining, which can be compared with actual amount of drug remaining to access accuracy of pump function, but actual pump function over time is not recorded. The pump is usually implanted in the right or left abdominal wall. Other pumps useful in the invention include, for example, portable disposable infuser pumps (PDIPs). Additionally, implantable infusion devices may employ liposome delivery systems, such as small unilamellar vesicles, large unilamellar vesicles, and multilamellar vesicles can be formed from a variety of phospholipids, such as cholesterol, stearyl amine or phosphatidylcholines.

The invention also includes delayed-release ocular preparations containing one or more glyponectins, glyponectin agonists, adiponectins, and/or adiponectin agonists, for example, one or more glycoisoforms and/or non-glycosylated isoforms. One of the problems associated with the use of ophthalmic solutions is the rapid loss of administered drug due to blinking of the eye and the flushing effect of lacrimal fluids. Up to 80% of an administered dose may be lost through tears and the action of nasolacrimal drainage within 5 minutes of installation. Extended periods of therapy may be achieved by formulations of the invention that increase the contact time between the glyponectin, glyponectin agonist, adiponectin, and/or adiponectin agonist and the corneal surface. This may be accomplished through use of agents that increase the viscosity of solutions; by ophthalmic suspensions in which the glyponectin, glyponectin agonist, adiponectin, and/or adiponectin agonist particles slowly dissolve; by slowly dissipating ophthalmic ointments; or by use of ophthalmic inserts. Preparations of one or more glyponectins, glyponectin agonists, adiponectins, and/or adiponectin agonists, for example, one or more glycoisoforms and/or non-glycosylated isoforms, suitable for ocular administration to humans may be formulated using synthetic high molecular weight cross-linked polymers such as those of acrylic acid (e.g., Carbopol 940) or gellan gum (Gelrite; see, Merck Index 12th Ed., 4389), a compound that forms a gel upon contact with the precorneal tear film (e.g. as employed in Timoptic-XE by Merck, Inc.).

Further examples include delayed-release ocular preparations containing glyponectin, glyponectin agonist, adiponectin, and/or adiponectin agonist in ophthalmic inserts, such as the OCUSERT system (Alza Inc.). Typically, such inserts are elliptical with dimensions of about 13.4 mm by 5.4 mm by 0.3 mm (thickness). The insert is flexible and has a glyponectin-, glyponectin agonist-, adiponectin-, and/or adiponectin agonist-containing core surrounded on each side by a layer of hydrophobic ethylene/vinyl acetate copolymer membranes through which the glyponectin, glyponectin agonist, adiponectin, and/or adiponectin agonist diffuses at a constant rate. The white margin around such devices contains white titanium dioxide, an inert compound that confers visibility. The rate of glyponectin, glyponectin agonist, adiponectin, and/or adiponectin agonist diffusion is controlled by the polymer composition, the membrane thickness, and the glyponectin, glyponectin agonist, adiponectin, and/or adiponectin agonist solubility. During the first few hours after insertion, the glyponectin, glyponectin agonist, adiponectin, and/or adiponectin agonist release rate is greater than that which occurs thereafter in order to achieve initially therapeutic glyponectin, glyponectin agonist, adiponectin, and/or adiponectin agonist levels. The glyponectin-, glyponectin agonist-, adiponectin-, and/or adiponectin agonist-containing inserts may be placed in the conjunctival sac from which they release their medication over a treatment period. Another form of an ophthalmic insert is a rod shaped, water-soluble structure composed of hydroxypropyl cellulose in which glyponectin, glyponectin agonist, adiponectin, and/or adiponectin agonist is embedded. The insert is placed into the inferior cul-de-sac of the eye once or twice daily as required for therapeutic efficacy. The inserts soften and slowly dissolve, releasing the glyponectin, glyponectin agonist, adiponectin, and/or adiponectin agonist that is then taken up by the ocular fluids. A further example of such a device is that furnished by Lacrisert (Merck Inc.).

The invention also provides in part dose delivery formulations and devices formulated to enhance bioavailability of glyponectin, glyponectin agonist, adiponectin, and/or adiponectin agonist. This may be in addition to or in combination with any of the formulations or devices described above.

Despite good hydrosolubility, one or more glyponectins, glyponectin agonists, adiponectins, and/or adiponectin agonists, such as glycoisoforms and/or non-glycosylated isoforms, may be poorly absorbed in the digestive tract. A therapeutically effective amount of glyponectin, glyponectin agonist, adiponectin, and/or adiponectin agonist is an amount capable of providing an appropriate level of glyponectin, glyponectin agonist, adiponectin, and/or adiponectin agonist in the bloodstream. By increasing the bioavailability of glyponectin, glyponectin agonist, adiponectin, and/or adiponectin agonist, a therapeutically effective level of glyponectin, glyponectin agonist, adiponectin, and/or adiponectin agonist may be achieved by administering lower dosages than would otherwise be necessary.

An increase in bioavailability of glyponectin, glyponectin agonist, adiponectin, and/or adiponectin agonist may be achieved by complexation of glyponectin, glyponectin agonist, adiponectin, and/or adiponectin agonist with one or more bioavailability or absorption enhancing agents or in bioavailability or absorption enhancing formulations.

The invention in part provides for the formulation of glyponectins, glyponectin agonists, adiponectins, and/or adiponectin agonists, for example, one or more glycoisoforms and/or non-glycosylated isoforms, with other agents useful to enhance bioavailability or absorption. Such bioavailability or absorption enhancing agents include, but are not limited to, various surfactants such as various triglycerides, such as from butter oil, monoglycerides, such as of stearic acid and vegetable oils, esters thereof, esters of fatty acids, propylene glycol esters, the polysorbates, sodium lauryl sulfate, sorbitan esters, sodium sulfosuccinate, among other compounds. By altering the surfactant properties of the delivery vehicle it is possible to, for example, allow a glyponectin, glyponectin agonist, adiponectin, and/or adiponectin agonist to have greater intestinal contact over a longer period of time that increases uptake and reduces side effects. Further examples of such agents include carrier molecules such as cyclodextrin and derivatives thereof, well known in the art for their potential as complexation agents capable of altering the physicochemical attributes of drug molecules. For example, cyclodextrins may stabilize (both thermally and oxidatively), reduce the volatility of, and alter the solubility of, the glyponectin, glyponectin agonist, adiponectin, and/or adiponectin agonist with which they are complexed. Cyclodextrins are cyclic molecules composed of glucopyranose ring units that form toroidal structures. The interior of the cyclodextrin molecule is hydrophobic and the exterior is hydrophilic, making the cyclodextrin molecule water-soluble. The degree of solubility can be altered through substitution of the hydroxyl groups on the exterior of the cyclodextrin. Similarly, the hydrophobicity of the interior can be altered through substitution, though generally the hydrophobic nature of the interior allows accommodation of relatively hydrophobic guests within the cavity. Accommodation of one molecule within another is known as complexation and the resulting product is referred to as an inclusion complex. Examples of cyclodextrin derivatives include sulfobutylcyclodextrin, maltosylcyclodextrin, hydroxypropylcyclodextrin, and salts thereof. Complexation of glyponectin, glyponectin agonist, adiponectin, and/or adiponectin agonist with a carrier molecule such as cyclodextrin to form an inclusion complex may thereby reduce the size of the glyponectin, glyponectin agonist, adiponectin, and/or adiponectin agonist dose needed for therapeutic efficacy by enhancing the bioavailability of the administered active agent.

The invention in part also provides for the formulation of glyponectins, glyponectin agonists, adiponectins, and/or adiponectin agonists, for example, one or more glycoisoforms and/or non-glycosylated isoforms, in a microemulsion to enhance bioavailability. A microemulsion is a fluid and stable homogeneous solution composed of four major constituents, respectively, a hydrophilic phase, a lipophilic phase, at least one surfactant (SA) and at least one cosurfactant (CoSA). A surfactant is a chemical compound possessing two groups, the first polar or ionic, which has a great affinity for water, the second which contains a longer or shorter aliphatic chain and is hydrophobic. These chemical compounds having marked hydrophilic character are intended to cause the formation of micelles in aqueous or oily solution. Examples of suitable surfactants include mono-, di- and triglycerides and polyethylene glycol (PEG) mono- and diesters. A cosurfactant, also sometimes known as “co-surface-active agent”, is a chemical compound having hydrophobic character, intended to cause the mutual solubilization of the aqueous and oily phases in a microemulsion. Examples of suitable co-surfactants include ethyl diglycol, lauric esters of propylene glycol, oleic esters of polyglycerol, and related compounds.

The invention in part also provides for the formulation of glyponectin, glyponectin agonist, adiponectin, and/or adiponectin agonist with various polymers to enhance bioavailability by increasing adhesion to mucosal surfaces, by decreasing the rate of degradation by hydrolysis or enzymatic degradation of the glyponectin, glyponectin agonist, adiponectin, and/or adiponectin agonist, and by increasing the surface area of the glyponectin, glyponectin agonist, adiponectin, and/or adiponectin agonist relative to the size of the particle. Suitable polymers can be natural or synthetic, and can be biodegradable or non-biodegradable. Delivery of low molecular weight active agents, such as for example glyponectins, glyponectin agonists, adiponectins, and/or adiponectin agonists, including glycoisoforms and/or non-glycosylated isoforms, may occur by either diffusion or degredation of the polymeric system. Representative natural polymers include proteins such as zein, modified zein, casein, gelatin, gluten, serum albumin, and collagen, polysaccharides such as cellulose, dextrans, and polyhyaluronic acid. Synthetic polymers are generally preferred due to the better characterization of degradation and release profiles. Representative synthetic polymers include polyphosphazenes, poly(vinyl alcohols), polyamides, polycarbonates, polyacrylates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Examples of suitable polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate). Synthetically modified natural polymers include cellulose derivatives such as alkyl celluloses, hydroxyalkyl celluloses, cellulose ethers, cellulose esters, and nitrocelluloses. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt. Each of the polymers described above can be obtained from commercial sources such as Sigma Chemical Co., St. Louis, Mo., Polysciences, Warrenton, Pa., Aldrich Chemical Co., Milwaukee, Wis., Fluka, Ronkonkoma, N.Y., and BioRad, Richmond, Calif. or can be synthesized from monomers obtained from these suppliers using standard techniques. The polymers described above can be separately characterized as biodegradable, non-biodegradable, and bioadhesive polymers, as discussed in more detail below. Representative synthetic degradable polymers include polyhydroxy acids such as polylactides, polyglycolides and copolymers thereof, poly(ethylene terephthalate), poly(butic acid), poly(valeric acid), poly(lactide-co-caprolactone), polyanhydrides, polyorthoesters and blends and copolymers thereof. Representative natural biodegradable polymers include polysaccharides such as alginate, dextran, cellulose, collagen, and chemical derivatives thereof (substitutions, additions of chemical groups, for example, alkyl, alkylene, hydroxylations, oxidations, and other modifications routinely made by those skilled in the art), and proteins such as albumin, zein and copolymers and blends thereof, alone or in combination with synthetic polymers. In general, these materials degrade either by enzymatic hydrolysis or exposure to water in vivo, by surface or bulk erosion. Examples of non-biodegradable polymers include ethylene vinyl acetate, poly(meth)acrylic acid, polyamides, polyethylene, polypropylene, polystyrene, polyvinyl chloride, polyvinylphenol, and copolymers and mixtures thereof. Hydrophilic polymers and hydrogels tend to have bioadhesive properties. Hydrophilic polymers that contain carboxylic groups (e.g., poly[acrylic acid]) tend to exhibit the best bioadhesive properties. Polymers with the highest concentrations of carboxylic groups are preferred when bioadhesiveness on soft tissues is desired. Various cellulose derivatives, such as sodium alginate, carboxymethylcellulose, hydroxymethylcellulose and methylcellulose also have bioadhesive properties. Some of these bioadhesive materials are water-soluble, while others are hydrogels. Polymers such as hydroxypropylmethylcellulose acetate succinate (HPMCAS), cellulose acetate trimellitate (CAT), cellulose acetate phthalate (CAP), hydroxypropylcellulose acetate phthalate (HPCAP), hydroxypropylmethylcellulose acetate phthalate (HPMCAP), and methylcellulose acetate phthalate (MCAP) may be utilized to enhance the bioavailability of the glyponectin, glyponectin agonist, adiponectin, and/or adiponectin agonist with which they are complexed. Rapidly bioerodible polymers such as poly(lactide-co-glycolide), polyanhydrides, and polyorthoesters, whose carboxylic groups are exposed on the external surface as their smooth surface erodes, can also be used for bioadhesive glyponectin, glyponectin agonist, adiponectin, and/or adiponectin agonist delivery systems. In addition, polymers containing labile bonds, such as polyanhydrides and polyesters, are well known for their hydrolytic reactivity. Their hydrolytic degradation rates can generally be altered by simple changes in the polymer backbone. Upon degradation, these materials also expose carboxylic groups on their external surface, and accordingly, these can also be used for bioadhesive glyponectin, glyponectin agonist, adiponectin, and/or adiponectin agonist delivery systems.

Other agents that may enhance bioavailability or absorption of one or more glyponectins, glyponectin agonists, adiponectins, and/or adiponectin agonists, for example, one or more glycoisoforms and/or non-glycosylated isoforms can act by facilitating or inhibiting transport across the intestinal mucosa. For example, it has long been suggested that blood flow in the stomach and intestine is a factor in determining intestinal drug absorption and drug bioavailability, so that agents that increase blood flow, such as vasodilators, may increase the rate of absorption of orally administered glyponectin, glyponectin agonist, adiponectin, and/or adiponectin agonist by increasing the blood flow to the gastrointestinal tract. Vasodilators have been used in combination with other drugs. For example, in EPO Publication 106335, the use of a coronary vasodilator, diltiazem, is reported to increase oral bioavailability of drugs which have an absolute bioavailability of not more than 20%, such as adrenergic beta-blocking agents (e.g., propranolol), catecholamines (e.g., dopamine), benzodiazepine derivatives (e.g., diazepam), vasodilators (e.g., isosorbide dinitrate, nitroglycerin or amyl nitrite), cardiotonics or antidiabetic agents, bronchodilators (e.g., tetrahydroisoquinoline), hemostatics (e.g., carbazochrome sulfonic acid), antispasmodics (e.g., timepidium halide) and antitussives (e.g., tipepidine). Vasodilators therefore constitute another class of agents that may enhance the bioavailability of glyponectin, glyponectin agonist, adiponectin, and/or adiponectin agonist.

Other mechanisms of enhancing bioavailability of the compositions and formulations of the invention include the inhibition of reverse active transport mechanisms. For example, it is now thought that one of the active transport mechanisms present in the intestinal epithelial cells is p-glycoprotein transport mechanism which facilitates the reverse transport of substances, which have diffused or have been transported inside the epithelial cell, back into the lumen of the intestine. It has been speculated that the p-glycoprotein present in the intestinal epithelial cells may function as a protective reverse pump which prevents toxic substances which have been ingested and diffused or transported into the epithelial cell from being absorbed into the circulatory system and becoming bioavailable. One of the unfortunate aspects of the function of the p-glycoprotein in the intestinal cell however is that it can also function to prevent bioavailability of substances which are beneficial, such as certain drugs which happen to be substrates for the p-glycoprotein reverse transport system. Inhibition of this p-glycoprotein mediated active transport system will cause less drug to be transported back into the lumen and will thus increase the net drug transport across the gut epithelium and will increase the amount of drug ultimately available in the blood. Various p-glycoprotein inhibitors are well known and appreciated in the art. These include, water-soluble vitamin E; polyethylene glycol; poloxamers including Pluronic F-68; Polyethylene oxide; polyoxyethylene castor oil derivatives including Cremophor EL and Cremophor RH 40; Chrysin, (+)-Taxifolin; Naringenin; Diosmin; Quercetin; and the like. Inhibition of a reverse active transport system of which, for example, glyponectin, glyponectin agonist, adiponectin, and/or adiponectin agonist is a substrate may thereby enhance the bioavailability of said glyponectin, glyponectin agonist, adiponectin, and/or adiponectin agonist.

The particular dosage regimen, which includes dose and frequency of administration, will depend on the particular subject and that subject's medical history. The treatment may include multiple administrations over a period of time. A treatment plan generally includes the administration of an effective amount of a composition of glyponectin, adiponectin and/or fragment, analog, etc. thereof, to the subject being treated. An effective amount can be determined by assessing biological activity, for example. A skilled artisan may determine the amount of a glycosylated composition by stepwise increments of dosage and assessing biological function at each step. In one embodiment the dosage form and/or therapeutically effective amount is an amount effective to elicit a plasma glyponectin and/or adiponectin concentration from about 1 μg/mL to about 20 μg/mL. In another embodiment the dosage form and/or therapeutically effective amount is an amount effective to elicit a plasma adiponectin concentration from about 1.9 μg/mL to about 17 μg/mL.

Any such dose may be administered by any of the routes or in any of the forms herein described. It will be appreciated that any of the dosage forms, compositions, formulations or devices described herein particularly for oral administration may be utilized, where applicable or desirable, in a dosage form, composition, formulation or device for administration by any of the other routes herein contemplated or commonly employed. For example, a dose or doses could be given parenterally using a dosage form suitable for parenteral administration which may incorporate features or compositions described in respect of dosage forms suitable for oral administration, or be delivered in an oral dosage form such as a modified release, extended release, delayed release, slow release or repeat action oral dosage form.

A better understanding of the invention will be gained by reference to the following experimental section. The following experiments are illustrative and are not intended to limit the invention or the claims in any way.

### EXAMPLE 1

### Glyponectin and Adiponectin Isolated from Mammalian Cells

Differentiation of 3T3-L1 cells and concentration of proteins from the cell culture medium—3T3-L1 cells were maintained as subconfluent cultures in Dulbecco's modified Eagle's medium (DMEM) supplemented with 10% fetal calf serum. For differentiation, cells were seeded onto 150 mm plates and allowed to reach 100% confluence. The cells were induced one-day post confluence with DMEM containing 0.25 μM dexamethasone, 0.5 mM 3-isobutyl-1-methylxanthine (IBMX) and 10 g/ml insulin for 2 days. This was followed by incubation with 10 μg/ml insulin for 2 days. The cells were then maintained in DMEM with 10% fetal calf serum for another 4 days.

To harvest proteins secreted from adipocytes, the cells at day 8 after differentiation were washed three times with phosphate-buffered saline (PBS), and then incubated with serum-free medium for another 4 hours. The medium was collected, centrifuged at 3,000×g for 10 minutes, filtered through 0.20 μm filter, and then concentrated and desalted using a concentrator with molecular mass cutoff of 5000 Da (Vivascience Ltd, Gloucestershire, UK). The proteins were then quantitated using BCA reagent, and stored at −80° C. until use.

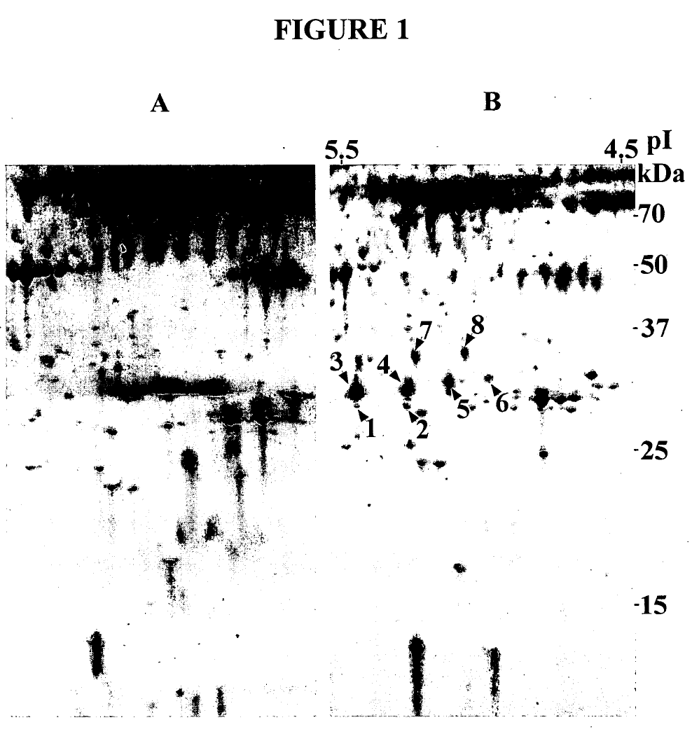

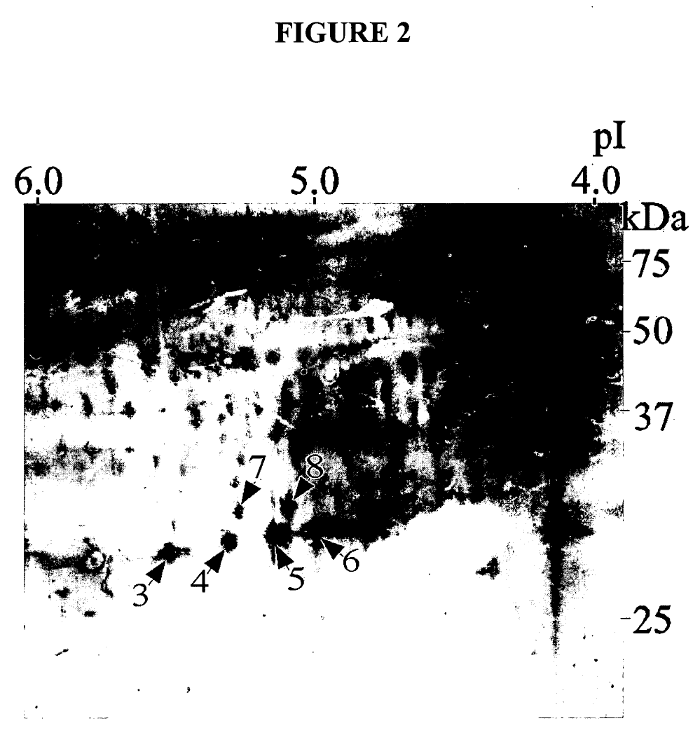

The proteins secreted from either adipocytes or 3T3 L1 preadipocytes were separated by two-dimensional gel electrophoresis (2-DE) as described in Wang et al., (2001) Diabetes 50, 1821-1827. The separated proteins were stained with either silver or Coomassie Brilliant Blue R250 (CBB). For immunoblotting, proteins separated by 2-DE were transferred to nitrocellulose membranes using a Multiphor II Novablot electrophoretic transfer unit (Pharmacia). The membranes were blocked, and then incubated overnight at 4° C. with rabbit anti-adiponectin polyclonal antibody (1:1000). After incubation with horseradish-peroxidase conjugated secondary antibody for one hour at room temperature, the bound antibodies were detected by enhance chemiluminescence (ECL) detection system (Roche). 8 proteins spots were detected in 3T3-L1 adipocytes, but not in undifferentiated 3T3-L1 cells. (See FIG. 1) Glycosylated proteins were detected using an Immun-Blot kit (Bio-Rad) according to the manufacturer's instructions. 6 of the 8 spots were glycosylated isoforms (glycoisoforms) of the protein. (See FIG. 2) Treatment with tunicamycin, an inhibitor of N-linked glycosylation, did not affect the glycosylation pattern. (Data not shown)

Amino acid sequencing was performed on the 8 isoforms. Protein spots separated by 2-DE above were transferred to polyvinylidene difluoride (PVDF) membrane, stained with Coomassie Brilliant Blue 250, excised, and subjected to amino acid sequencing using the Edman degradation method with a Perkin-Elmer (Procise, Model 492) protein sequencer. N-terminal amino acids match the amino acid residues 18-25 of mouse adiponectin.

The 8 isoforms were then trypsin digested and fractionated by Reversed-phase High Performance Liquid Chromatography (RP-HPLC). Briefly, the 8 protein spots were excised, and gel pieces were subjected to in-gel trypsin digestion as described in Wang et al., (1999) FEBS Lett. 462, 25-30.

The extracted tryptic peptide mixtures were fractionated by RP HPLC on a Jupiter 5μ. C18 column (250×2.00 mm, Phenomenex). The pre-warmed column (37° C.) was washed for 7 min with 0.1% trifluoroacetic acid (v/v), followed by elution using a 50-min linear gradient from 8% to 36% of acetonitrile at the flow rate of 200 μl/min. Each fraction was collected manually and subjected to further analysis as described below. Three tryptic fragments were isolated and designate A-C.

Internal amino acid sequences were obtained by sequencing the tryptic peptides following RP-HPLC fractionation. Three protein trypsin fractions (A-C) were sequenced and identified as amino acid fragments corresponding to amino acid residues 62-95 (B & C) and 104-115 (A) of mouse adiponectin. The amino acids of these fragments were all identified, except for the four lysine residues (residues 68, 71, 80 and 104 in mouse adiponectin). Thus these lysines residues are believed to be modified by the addition of sugar group.

For amino acid analysis, 5 μg of the tryptic peptides were vacuum dried and hydrolyzed in the gas phase with 6 M HCl and 1% phenol for 24 hours at 110° C. This treatment destroyed sugar residues but still permitted detection and quantitation of hydroxylysine and hydroxyproline (Johnson et. al., (1980)_Clin. Orthop. Relat. Res. 282-288. Free amino acid residues were dissolved in 40 μl of 0.025% K₃EDTA, derivatized with phenylisothiocyanate (PITC), separated on a Spheri-5 PTC 5μ column (220×2.1 mm) and analyzed by 421 amino acid analyzer (Applied Biosystems Model 421).

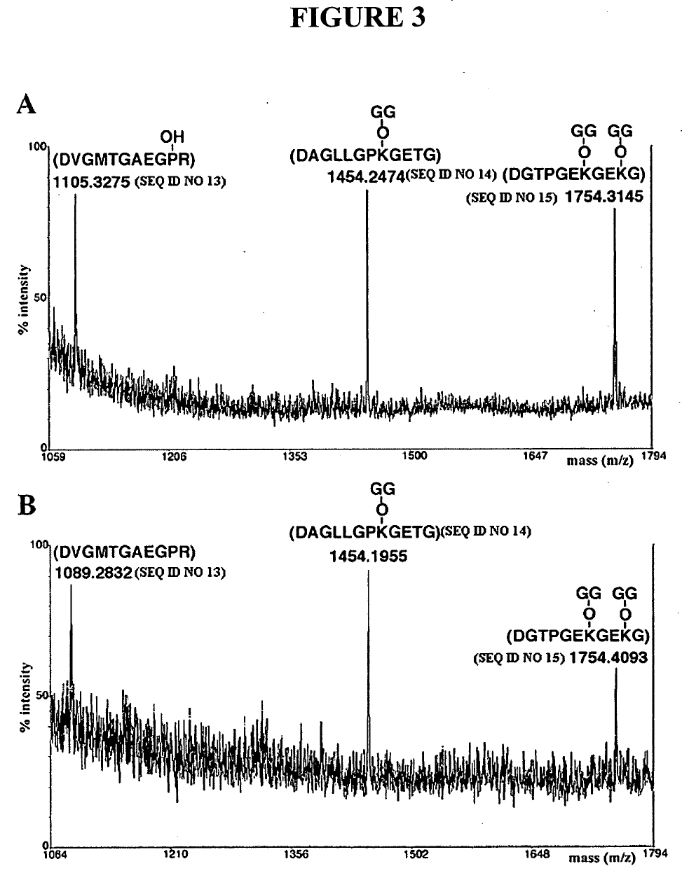

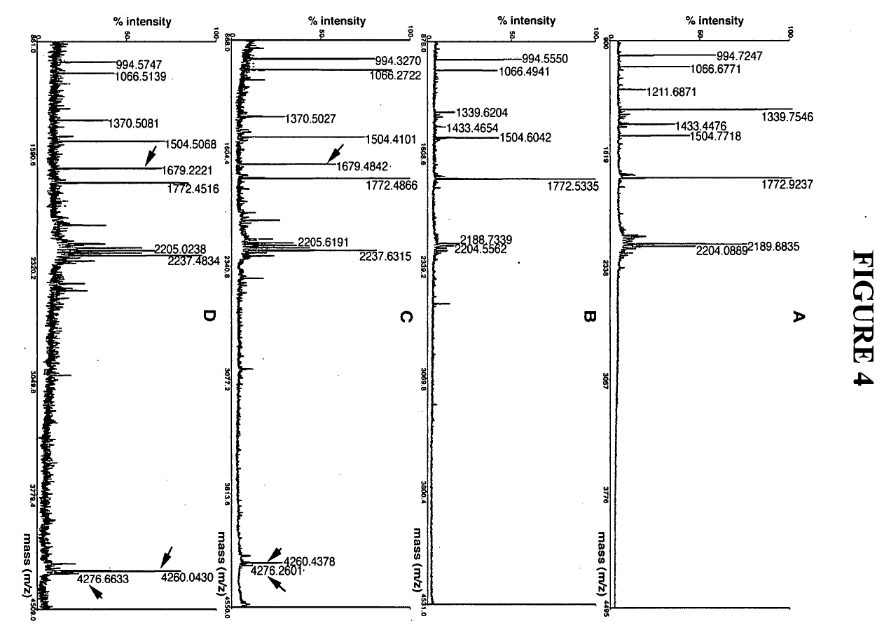

The mass spectra of these treated fragments were then analyzed using matrix-assisted laser desorption ionization time of flight mass spectrometry (MALDI-TOF MS). Briefly, 0.5 μl of the tryptic peptide mixtures or RP-HPLC separated peptides was mixed with an equal amount of α-cyano-4-hydroxycinnamic acid matrix (10 mg/ml in 60% acetonitril and 0.3% TFA), spotted onto the sample plates and air-dried. Reflectron mass spectrometric analyses were performed on a Voyager DE PRO Biospectrometry Workstation (Applied Biosystems) using a pulsed laser beam (nitrogen laser, λ=337 nm). All ion spectra were recorded in the positive mode with the accelerating voltage of 20.0 kV. The spectrometer was externally calibrated using Cal Mix 2 standard mixture. The experimental mass of peptide B (containing three lysine residues) differed from its theroretical mass. (See FIG. 3) This difference in mass is the expected size for the three glucosylgalatosylhydroxyl groups. Additionally, FIG. 4 shows the MALDI-TOF MS analysis of (A) bacterially produced, recombinant glyponectin; (B) isoform 1 of glyponectin secreted from 3T3-L1 adipocytes; (C) isoform 3 of glyponectin secreted from 3T3-L1 adipocytes; and (D) isoform 3 of recombinant glyponectin expressed in COS-7 cells. The peptides with masses of 1679, 4260 and 4276 Da are reproducibly observed in all the glycoisoforms produced in both adipocytes and COS-7 cells, but not seen in isoform 1, isoform 2 or bacterially expressed recombinant proteins.

### EXAMPLE 2

### Recombinant Glyponectin Polypeptide Expressed in Mammalian Cells

Glyponectin was recombinantely produced by isolating total RNA from 3T3-L1 adipocytes using Trizol reagent according to the manufacturer's instructions. The oligo-dT-primed cDNA from the total RNA was used as a template for PCR cloning based on mouse adiponectin nucleotide sequence (accession number: U37222). Full-length cDNA of adiponectin was then inserted into pGEMT-easy vector and its sequence was verified by DNA sequencing. The protein sequence was counted starting from the methionine residue.

To prepare a prokaryotic expression plasmid for mouse adiponectin, the cDNA sequence was amplified using 5′ATCGGGATCCGAAGATGACGTTACTACAACT3′ (SEQ ID NO 8) as the sense primer and 5′TACGAATTCTCAGTTGGTATCATGGTAGAG3′ (SEQ ID NO 9)as the antisense primer. The BamHI/SalI fragment of the amplified DNA product was subcloned into pPROEX HTb plasmid, resulting in expression vector pPRO-His-Ad that encodes full-length adiponectin with a His₆ tag (SEQ ID NO: 24) at its N-terminus. A similar strategy was used for the construction of a prokaryotic expression vector pPRO-His-gAd, which expresses His₆-tagged globular region of adiponectin (amino acid residues between 110 and 247), except that the sense primer is 5′ATCGGGATCCGCCGCTTATATGTATCGCTC3′ (SEQ ID NO 10). The expression of His-tagged full-length adiponectin or its globular region in BL21 cells was induced by the addition of 1 mM of isopropyl-p-D-thiogalactopyranoside into the growth medium. Full-length adiponectin or its globular region was purified from the bacterial lysates using Ni²⁺-NTA-agarose column according to the manufacturer's instructions. Following purification, the N-terminal tag was removed by cleavage with recombinant TEV protease. The purity of the protein was confirmed by SDS-PAGE and HPLC.

The vector for mammalian expression of glyponectin was generated by cDNA amplification using 5′GCCCGCGGATCCATGCTACTGTTGCAAGCTCT3′ (SEQ ID NO 11) as the sense primer and 5′GGCCGCGAATTCTCACTTGTCATCGTCGTCCTTGTAGTCG TTGGTATCATGGTAGAG3′ (SEQ ID NO 12) as the antisense primer. Following digestion with BamHI/EcoRI, the fragment was inserted into pcDNA3.1(+) vector to produce expression vector pcDNA-Ad-F, which encodes full-length glyponectin with FLAG epitope tagged at its C-terminus.

This expression vector was then used as a template to construct the vectors encoding glyponectin variants in which the four lysines (68, 71, 80 and 104) were replaced by arginines, using a QuikChange site-directed mutagenesis kit. The mutagenic oligonucleotide primers were designed according to the criteria recommended by the manufacturer, with the codon changes from AAG to CGG (for 68 and 80) or from AAA to CGA (for 71 and 104). A plasmid (named as pcDNA-Ad (K→R)-F), encoding a FLAG-tagged glyponectin variant with all the four lysines substituted with arginines was obtained by sequential mutation of each site, and all the mutations were confirmed by DNA sequencing.

These mammalian expression vectors were transfected into COS-7 cells using FuGENE 6 transfection reagent, and the cells were allowed to secrete glyponectin into serum free medium for 48 hours. The medium was then harvested and precipitated by incubation with 40% ammonium sulfate overnight at 4° C. After subsequent centrifugation at 8,000×g for 1 hour, the pellets were resuspended in TBS, dialysed against the same buffer using SnakeSkin tube with MWCO of 7000 Da. FLAG-tagged glyponectin was purified using anti-FLAG M2 affinity Gel, and eluted with 150 μg/ml of FLAG peptide. 2-DE analysis of recombinant glyponectin showed 8 protein spots, with a pattern similar to that seen in the glyponectin isolated from the 3T3-L1 adipocytes.

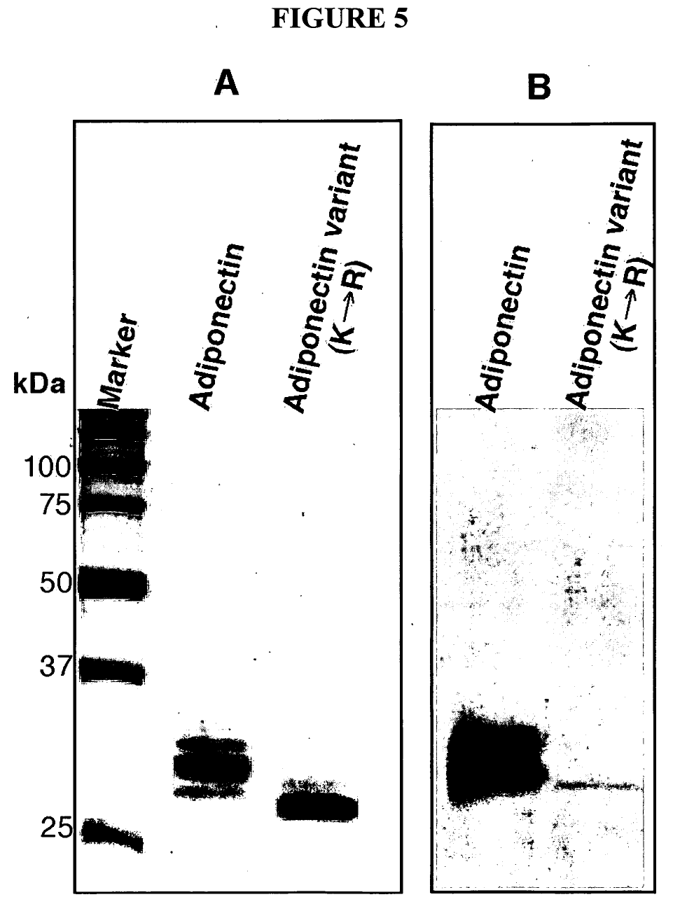

The recombinant adiponectin expressed in E. coli, COS-7 and the COS-7 expressed arganine mutated glyponectin were all run out on SDS-PAGE gel. The adiponectin expressed in E. coli had only a single band. The recombinant glyponectin expressed in COS-7 cells had three bands: one band the size of the E. coli produced adiponectin and two larger bands, the larger bands being glycoisoforms. The glyponectin with all four lysines mutated to arganines showed only a single band, the same size as the adiponectin expressed in E. Coli, E. coli are not capable of glycosylating proteins, and this single band of protein provides further evidence that these lysine residues are glycosylated. (See FIG. 5)

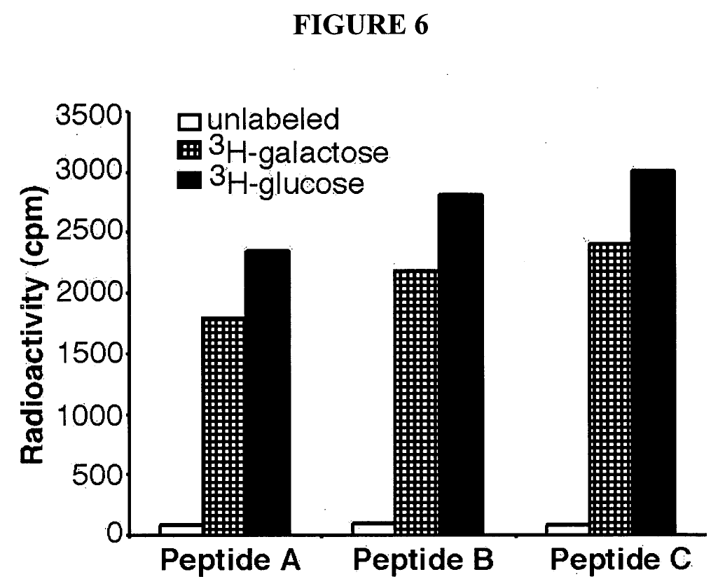

To confirm the presence of galactose and glucose moeities, COS-7 cells were transfected with recombinant glyponectin and radiolabeled with 100 μCi/ml [1-3H]galactose in DMEM or with [1-3H]glucose in DMEM containing 2 mM galactose for 48 hours. FLAG-tagged glyponectin was purified from the cell medium and tryptic peptide mixtures were separated by RP-HPLC to obtain peptides A-C as described above. The fractions containing each peptide were subjected to liquid scintillation counting. Results show that galactose and glucose was incorporated into all three peptides. (See FIG. 6)

### Example 3

### In Vivo Effects of Glyponectin in Rats

The effects of glyponectin on mice fed a modified high-fat liquid ethanol treatment diet were evaluated. The experiment included evaluation of baseline physiological data from high-fat liquid control diets in mice. An Institutional Animal Ethics Committee approved all experimental protocols used in this study.

Male FVB/n mice (n=15) weighing between 25-30 grams) were housed in stainless steel wire-bottom cages on a 12-hour light/12-hour dark cycle under the institutional guidelines for the humane treatment of laboratory animals.

Ten mice were fed with a modified high-fat/low carbohydrate liquid ethanol (LE) diet containing 44% fat, 16% protein, 5.5% carbohydrate, plus 34.5% ethanol after week one. During week one the ethanol concentration was gradually increased from 17% to 34% and then maintained at this concentration for another 5 weeks.

The five remaining mice were fed with a modified high-fat/low carbohydrate liquid (LC) diet containing 44% fat, 16% protein, 5.5% carbohydrate, plus 34.5% isocaloric maltose dextrin that would provide the same caloric count as ethanol, as the control. The dietary intake of control groups was matched to that of the ethanol-fed group by pair-feeding the same volume of isocaloric liquid diet. Three weeks after being fed with the HF/LE diet, the mice from Example 1 were surgically implanted with an osmotic pump (Alzet, Newark, Del.) which delivered 30 μg/day of recombinant glyponectin, or physiological saline (control). Delivery of glyponectin at this dosage caused a 2.7.±0.0.3 fold increase in the circulating concentration of glyponectin over that of untreated LE mice.

Mice that were offered the LE diet as their sole food source consumed 15.0±1.3 ml/day/mouse. The amount of ethanol consumed was approximately 17-19 g/kg body weight per day. Observations of the effects of alcohol in mice included, but were not limited to, hyperkinetic behavior, aggression, agitation, disorientation and dyskinesia (i.e., repetitive involuntary purposeless movements). These behavioral effects were ameliorated within 24 hours of glyponectin administration. Observed amelioration of said behavioral effects occurred even though the mice continued to receive the LE diet.

From the foregoing, it will be appreciated that, although specific embodiments of the invention have been described herein for the purpose of illustration, various modifications may be made without deviating from the spirit and scope of the invention. Accordingly, the present invention is not limited except as by the appended claims.

All patents, patent applications, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Additionally, all claims in this application, and all priority applications, including but not limited to original claims, are hereby incorporated in their entirety into, and form a part of, the written description of the invention. Applicants reserve the right to physically incorporate into this specification any and all materials and information from any such patents, applications, publications, scientific articles, web sites, electronically available information, and other referenced materials or documents. Applicants reserve the right to physically incorporate into any part of this document, including any part of the written description, the claims referred to above including but not limited to any original claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features reported and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

I claim:

1. A method of treating an alcohol-related disease, disorder or condition of the mind and/or brain in a subject, comprising administering to said subject a composition comprising a therapeutically effective amount of a glyponectin and/or a glyponectin agonist.

2. A method of claim 1 wherein the disease, disorder or condition is related to excessive alcohol intake.

3. A method of claim 1 wherein the disease, disorder or condition is related to chronic alcohol intake.

4. A method of claim 1 wherein the disease, disorder or condition is related to chronic excessive alcohol intake.

5. A method of claim 1 wherein the alcohol-related disease, disorder or condition of the mind and/or brain is characterized at least in part by a behavioral irregularity.

6. A method of claim 5 wherein said behavioral irregularity is one or more of agitation, aggression, dyskinesis and hyperkinesis.

7. A method of claim 1 wherein the alcohol-related disease, disorder or condition of the mind and/or brain is characterized at least in part by cognitive difficulty.

8. A method of claim 7 wherein said cognitive difficulty is one or more of impaired memory function, deficits in problem solving, and complex thinking.

9. A method of claim 1 wherein the alcohol-related disease, disorder or condition of the mind and/or brain is selected from the group consisting of Wernicke's syndrome, and Korsakoff's syndrome.

10. A method of claim 1 wherein the alcohol-related disease, disorder or condition of the mind and/or brain is alcoholic cerebellar degeneration.

11. A method of claim 1 wherein the alcohol-related disease, disorder or condition of the mind and/or brain is alcohol-induced hypopituitarism.

12. A method of claim 1 wherein the alcohol-related disease, disorder or condition of the mind and/or brain is altered sleep stages as a result of alcohol intake.

13. A method of claim 12 wherein said altered sleep stages are any one or more of insomnia, delayed sleep onset, frequent awakenings, reduced amounts of nonREM sleep, sleep apnea or hypoxia.

14. A method of claim 1 wherein the alcohol-related disease, disorder or condition of the mind and/or brain is also characterized, at least in part, by alcoholic peripheral neuropathy.

15. A method of claim 1 wherein the alcohol-related disease, disorder or condition of the mind and/or brain is an alcohol-induced psychotic disorder.

16. A method of treating a subject for hallucinations comprising administering to said subject a composition comprising a therapeutically effective amount of a glyponectin and/or a glyponectin agonist.

17. A method of claim 16 wherein the hallucinations are alcohol-induced.

18. A method of treating a subject for paranoid delusions, comprising administering said subject a therapeutically effective amount of a glyponectin and/or a glyponectin agonist.

19. A method of claim 18 wherein the paranoid delusions are alcohol-induced.

20. A method of treating a subject for a mood disorder, comprising administering to said subject a therapeutically effective amount of a glyponectin and/or a glyponectin agonist.

21. A method of claim 20 wherein the mood disorder is alcohol-induced.

22. A method of claim 20 wherein the mood disorder is one or more of depression, sadness, or anxiety.

23. A method of treating a subject exhibiting an increased level and/or pattern of alcohol consumption, which comprises administering to said subject a therapeutically effective amount of a glyponectin and/or a glyponectin agonist.

24. A method of claim 23 wherein said subject exhibits any one or more of acute alcohol intoxication, chronic alcohol intoxication, alcohol abuse, alcoholism, or alcohol dependence.

25. A method of treating a subject for alcohol withdrawal syndrome, comprising administering to said subject a therapeutically effective amount of a glyponectin and/or a glyponectin agonist.

26. A method of claim 25 wherein said subject exhibits any one or more of irritability, agitation, tremors, delirium tremens, or seizures.

27. A method for treating a subject for an alcohol-induced digestive system disease, disorder or condition, comprising administering to said subject a therapeutically effective amount of a glyponectin and/or a glyponectin agonist.

28. A method of claim 27 wherein the digestive system disease, disorder or condition is any one or more of esophagitis, acute gastritis, chronic gastritis, acute pancreatitis and chronic pancreatitis.

29. A method of treating a subject for an alcohol-induced cardiovascular disease, disorder or condition comprising administering to said subject a therapeutically effective amount of a glyponectin and/or a glyponectin agonist.

30. A method of claim 29 wherein the alcohol-induced cardiovascular disease, disorder or condition is any one or more of alcoholic cardiomyopathy, alcohol-induced cardiac arrhythmia, hypertension, coronary artery disease, or stroke.

31. A method of treating a subject for alcoholic myopathy, comprising administering to said subject a therapeutically effective amount of a glyponectin and/or a glyponectin agonist.

32. A method of treating a subject for extrapulmonary tuberculosis, comprising administering to said subject a therapeutically effective amount of a glyponectin and/or a glyponectin agonist.

33. A method of any one of claims 1, 16, 18, 20, 23, 25, 27, 29, 31, or 32 wherein the glyponectin is selected from the group consisting of recombinant glyponectin, human glyponectin, and recombinant human glyponectin.

34. A method of claim 33 wherein the subject is human.

35. A method of claim 33 wherein the glyponectin is at least about 50% pure, at least about 80% pure, at least about 90% pure, at least about 95% pure, or at least about 99% pure.

36. A method of claim 33 wherein said therapeutically effective amount is the amount effective to elicit a plasma concentration from about 1 μg/mL to about 20 μg/mL.

37. A method of claim 33 wherein the glyponectin is selected from the group consisting of a glyponectin comprising one or more glycosylated lysine residues, a glyponectin comprising two or more glycosylated lysine residues, and a glyponectin comprising three or more glycosylated lysine residues.

38. A method of claim 33 wherein the glyponectin is selected from the group consisting of a glyponectin comprising one or more glycosylated lysine residues, a glyponectin comprising two or more glycosylated lysine residues, and a glyponectin comprising three or more glycosylated lysine residues, and wherein the lysine residue is one or more of the lysine residues corresponding to positions 65, 68, 77, and 101 of human glyponectin.

39. A method of claim 33 wherein the glyponectin is selected from the group consisting of a glyponectin comprising one or more glycosylated lysine residues, a glyponectin comprising two or more glycosylated lysine residues, and a glyponectin comprising three or more glycosylated lysine residues, wherein the lysine residue is one or more of the lysine residues corresponding to positions 65, 68, 77, and 101 of human glyponectin, and wherein one or more of the lysine residues comprise a glucosylgalactosyl moiety, a glucosylglucosyl moiety, a galactosylglucosyl moiety, or a galactosylgalactosyl moiety.

40. A method of claim 39 wherein one or more of the lysine residues at positions 65, 68, 77, and 101 is α-1-2-glucosylgalactosyl-O-hydroxylysine.

41. A method of claim 33 wherein the glyponectin has the amino acid sequence of a naturally occurring human glyponectin.

42. A method of claim 33 wherein the glyponectin comprises a hydroxyproline at the amino acid residue corresponding to position 91 of human glyponectin.

43. A method of claim 33 wherein the glyponectin is a mixture of one or more glycoisoforms of human glyponectin.

44. A method of claim 33 wherein the composition is substantially free of non-glycosylated glyponectin.

45. A method of claim 33 wherein the glyponectin is formulated for parenteral administration.

46. A method of claim 45 wherein the parenteral administration is by subcutaneous, intradermal, intravenous, intraperitoneal, or transdermal administration.


SEQUENCE LISTING (24 sequences)

SEQ ID NO: 1 - DNA, 819 bp, Homo sapiens
aggctgttgaggctgggccatctcctcctcacttccattctgactgcagtctgtggttctgattccataccagaggggctcaggatgctgttgctgggagctgttctactgctattagctctgcccggtcatgaccaggaaaccacgactcaagggcccggagtcctgcttcccctgcccaagggggcctgcacaggttggatggcgggcatcccagggcatccgggccataatggggccccaggccgtgatggcagagatggcacccctggtgagaagggtgagaaaggagatccaggtcttattggtcctaagggagacatcggtgaaaccggagtacccggggctgaaggtccccgaggctttccgggaatccaaggcaggaaaggagaacctggagaaggtgcctatgtataccgctcagcattcagtgtgggattggagacttacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactga

SEQ ID NO: 2 - protein, 244 aa, Homo sapiens
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 3 - protein, 243 aa, Macaca mulatta
MLLGAVLLLLALPSHGQDTTTQGPGVLLPLPKGACTGWMAGIPGHPGHNGVPGRDGRDGTPGEKGEKGDPGLIGPKGDTGETGVTGAEGPRGFPGIQGRKGEPGEGAYVYRSAFSVGLETYVTVPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 4 - protein, 247 aa, Mus musculus
MLLLQALLFLLILPSHAEDDVTTTEELAPALVPPPKGTCAGWMAGIPGHPGHNGTPGRDGRDGTPGEKGEKGDAGLLGPKGETGDVGMTGAEGPRGFPGTPGRKGEPGEAAYMYRSAFSVGLETRVTVPNVPIRFTKIFYNQQNHYDGSTGKFYCNIPGLYYFSYHITVYMKDVKVSLFKKDKAVLFTYDQYQEKNVDQASGSVLLHLEVGDQVWLQVYGDGDHNGLYADNVNDSTFTGFLLYHDTN

SEQ ID NO: 5 - protein, 244 aa, Rattus sp.
MLLLQALLFLLILPSHEGITATEGPGALVPPPKETCAGWMAGIPGYPGHNGIPGRDGRDGTPGEKGEKGDAGVLGPKGDPGDAGMTGAEGPRGFPGTPGRKGEPGEAAYMYHSAFSVGLETRVTVPNVPIRFTKIFYNQQNHYDGSTGKFHCNIPGLYYFSYHITVYMKDVKVSLFKKDKAVLFTYDQYQEKNVDQASGSMLLHLEVGDQVWLQVYGEGDNNGLYADNVNDSTFTGFLLYHDTN

SEQ ID NO: 6 - protein, 244 aa, Canis familiaris
MLLLRAVLLLLVLPAHGQDSVAEGPGVLLPLPKGACPGWMAGIPGHPGHNGTPGRDGRDGTPGEKGEKGDAGLVGPKGDTGETGVTGVEGPRGFPGTPCRKGEPGESAYVHRSAFSVGLESRITVPNVPIRFTKIFYNLQNHYDGTTGKFHCNIPGLYYFSYHITVYLKDVKVSLYKKDKAMLFTYDQYQEKNVDQASGSVLLHLEVGDQVWLQVYGDGDSYGIYADNVNDSTFTGFLLYHDTN

SEQ ID NO: 7 - protein, 240 aa, Bos taurus
MLLQGALLLLLALPSHGEDNMEDPPLPKGACAGWMAGIPGHPGHNGTPGRDGRDGTPGEKGEKGDAGLLGPKGETGDVGMTGAEGPRGFPGTPGRKGEPGEAAYVYRSAFSVGLETRVTVPNVPIRFTKIFYNQQNHYDGSTGKFYCNIPGLYYFSYHITVYMKDVKVSLFKKDKAVLFTYDQYQEKNVDQASGSVLLHLEVGDQVWLQVYEGENHNGVYADNVNDSTFTGFLLYHNIVE

SEQ ID NO: 8 - DNA, 31 bp, Artificial Sequence
  Description of Artificial Sequence Synthetic Primer
atcgggatccgaagatgacgttactacaact

SEQ ID NO: 9 - DNA, 30 bp, Artificial Sequence
  Description of Artificial Sequence Synthetic Primer
tacgaattctcagttggtatcatggtagag

SEQ ID NO: 10 - DNA, 30 bp, Artificial Sequence
  Description of Artificial Sequence Synthetic Primer
atcgggatccgccgcttatatgtatcgctc

SEQ ID NO: 11 - DNA, 32 bp, Artificial Sequence
  Description of Artificial Sequence Synthetic Primer
gcccgcggatccatgctactgttgcaagctct

SEQ ID NO: 12 - DNA, 57 bp, Artificial Sequence
  Description of Artificial Sequence Synthetic Primer
ggccgcgaattctcacttgtcatcgtcgtccttgtagtcgttggtatcatggtagag

SEQ ID NO: 13 - protein, 11 aa, Artificial Sequence
  Description of Artificial Sequence Synthetic peptide
DVGMTGAEGPR

SEQ ID NO: 14 - protein, 12 aa, Artificial Sequence
  Description of Artificial Sequence Synthetic peptide
DAGLLGPKGETG

SEQ ID NO: 15 - protein, 11 aa, Artificial Sequence
  Description of Artificial Sequence Synthetic peptide
DGTPGEKGEKG

SEQ ID NO: 16 - protein, 8 aa, Artificial Sequence
  Description of Artificial Sequence Synthetic peptide
GEKGEKGD

SEQ ID NO: 17 - protein, 5 aa, Artificial Sequence
  Description of Artificial Sequence Synthetic peptide
GPKGE

SEQ ID NO: 18 - protein, 5 aa, Artificial Sequence
  Description of Artificial Sequence Synthetic peptide
GRKGE

SEQ ID NO: 19 - protein, 120 aa, Mus musculus
MLLLQALLFLLILPSHAEDDVTTTEELAPALVPPPKGTCAGWMAGIPGHPGHNGTPGRDGRDGTPGEKGEKGDAGLLGPKGETGDVGMTGAEGPRGFPGTPGRKGEPGEAAYVYRSAFSV

SEQ ID NO: 20 - protein, 117 aa, Homo sapiens
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSV

SEQ ID NO: 21 - protein, 112 aa, Bos taurus
MLLQGALLLLLALPSHGEDNMEDPPLPKGACAGWMAGIPGHPGHNGTPGRDGRDGTPGEKGEKGDAGLLGPKGETGDVGMTGAEGPRGFPGTPGRKGEPGEAAYVYRSAFSV

SEQ ID NO: 22 - protein, 116 aa, Macaca mulatta
MLLGAVLLLLALPSHGQDTTTQGPGVLLPLPKGACTGWMAGIPGHPGHNGVPGRDGRDGTPGEKGEKGDPGLIGPKGDTGETGVTGAEGPRGFPGIQGRKGEPGEGAYVYRSAFSV

SEQ ID NO: 23 - protein, 94 aa, Canis familiaris
GPGVLLPLPKGACPGWMAGIPGHPGHNGTPGRDGRDGTPGEKGEKGDPGLVGPKGDTGETGVTGVEGPRGFPGTPGRKGEPGESAYVHRSAFSV

SEQ ID NO: 24 - protein, 6 aa, Artificial Sequence
  Description of Artificial Sequence Synthetic 6-His tag
HHHHHH